(19) United States
(12) Patent Application Publication
(10) Pub. No.: US 2022/0169986 A1
(43) Pub. Date: Jun. 2, 2022

(54) MULTI-RESPIRATORY VIRUS ANTIGEN-SPECIFIC T CELLS AND METHODS OF MAKING AND USING THE SAME THERAPEUTICALLY

(71) Applicant: Baylor College of Medicine, Houston, TX (US)

(72) Inventors: Ann Marie Leen, Houston, TX (US); Juan Fernando Vera Valdes, Bellaire, TX (US)

(21) Appl. No.: 17/593,747

(22) PCT Filed: Mar. 25, 2020
(86) PCT No.: PCT/US2020/024726

§ 371 Date: Sep. 23, 2021

Related U.S. Application Data
(60) Provisional application No. 62/823,446, filed on Mar. 25, 2019.

(51) Int. Cl. C12N 5/0783; A61K 39/155; A61K 39/145; A61K 39/295; A61P 31/14; A61P 31/16

(52) U.S. Cl. CPC C12N 5/0638; A61K 39/155; A61K 39/145; A61K 39/295; A61P 31/14; A61P 31/16; A61K 2039/54; C12N 2760/16134; C12N 2760/18534; C12N 2760/18334; C12N 2501/2307; C12N 2501/2304; C12N 2760/18634

(57) ABSTRACT

Embodiments of the disclosure concern multi-respiratory vims specific T cell lines and methods of using the same to treat and prevent viral infections.

### CROSS-REFERENCE TO RELATED APPLICATIONS

This application claims priority to U.S. Provisional Application No. 62/823,446, filed Mar. 25, 2019, which application is incorporated by reference herein in its entirety

### TECHNICAL FIELD

Embodiments of the disclosure concern at least the fields of cell biology, molecular biology, immunology, and medicine.

### BACKGROUND

Viral infections are a serious cause of morbidity and mortality after allogenic hematopoietic stem cell transplantation (allo-HSCT), which is the treatment of choice for a variety of disorders. Post-transplant, however, graft versus host disease (GVHD), primary disease relapse and viral infections remain major causes of morbidity and mortality. Respiratory tract infections due to community-acquired respiratory viruses including respiratory syncytial virus, Influenza, parainfluenza virus and human metapneumovirus are detected in up to 40% of allogeneic hematopoietic stem cell transplant recipients in whom they cause severe symptoms including pneumonia and bronchiolitis and can be fatal. Other respiratory viruses including adenoviruses (AdV), rhinovirus and coronaviruses strains including SARS-CoV, SARS-CoV-2, MERS-CoV, and also the endemic CoVs that commonly afflict immunocompromised patients can also cause severe symptoms, especially in immunocompromised individuals, and the recent SARS-CoV2 pandemic has clearly exposed how ill-prepared we are to treat and prevent such infections. Given the lack of effective antivirals and the data from our group demonstrating that adoptively transferred ex vivo-expanded virus-specific T cells can be clinically beneficial for the treatment of both latent (Epstein-Barr virus, cytomegalovirus, BK virus, human herpesvirus 6) and lytic (adenovirus) viruses, we investigated the potential for extending this immunotherapeutic approach to respiratory viruses. Although available for some viruses, antiviral drugs are not always effective, highlighting the need for novel therapies. One strategy to treat these viral infections is with adoptive T cell transfer, whereby virus-specific T cells (VSTs) are expanded from the peripheral blood of healthy donors ex vivo and then infused to an individual with a viral infection, a stem cell transplant recipient, for example.

In vitro expanded donor-derived and third party virus-specific T cells targeting Adv, EBV, CMV, BK, HHV6 have shown to be safe when adoptively transferred to stem cell transplant patients with viral infections. Virus-specific T cells reconstituted antiviral immunity for Adv, EBV, CMV, BK and HHV6, were effective in clearing disease, and exhibited considerable expansion in vivo. Adoptively transferred in vitro expanded virus-specific T cells have also been shown to be safe and associated with clinical benefit when adoptively transferred to patients.

Embodiments of the present disclosure satisfy a long-felt need in the art by providing therapies for certain viruses by administering ex vivo-expanded, non-genetically modified, virus-specific T cells to control viral infection and ameliorate/eliminate one or more disease symptoms.

### SUMMARY OF THE EMBODIMENTS

In some embodiments, the present disclosure provides a composition comprising a polyclonal population of virus specific T-lymphocytes (VSTs) that recognize a plurality of viral antigens, wherein the plurality of viral antigens comprise at least one first antigen from PIV and at least one second antigen from one or more second viruses. In some embodiments, the VSTs are generated by contacting peripheral blood mononuclear cells (PBMCs) with a plurality of pepmix libraries, each pepmix library containing a plurality of overlapping peptides spanning at least a portion of a viral antigen, wherein at least one of the plurality of pepmix libraries spans a first antigen from PIV-3 and wherein at least one additional pepmix library of the plurality of pepmix libraries spans each second antigen. In some embodiments, the VSTs are generated by contacting T cells with dendritic cells (DCs) primed with a plurality of pepmix libraries, each pepmix library containing a plurality of overlapping peptides spanning at least a portion of a viral antigen, wherein at least one of the plurality of pepmix libraries spans a first antigen from PIV-3 and wherein at least one additional pepmix library of the plurality of pepmix libraries spans each second antigen. In some embodiments, the VSTs are generated by contacting T cells with dendritic cells (DCs) nucleofected with at least one DNA plasmid encoding the PIV-3 antigen and at least one DNA plasmid encoding each second antigen. In some embodiments, the plasmid encodes at least one PIV-3 antigen and at least one of the second antigens. In some embodiments, the VSTs comprise CD4+T-lymphocytes and CD8+T-lymphocytes. In some embodiments, the VSTs express αβ T cell receptors. In some embodiments, the VSTs comprise MHC-restricted T lymphocytes. In some embodiments, the one or more second viruses are selected from the group consisting of respiratory syncytial virus (RSV), Influenza, human metapneumovirus (hMPV) and a combination thereof. In some embodiments, the one or more second viruses comprise respiratory syncytial virus (RSV), Influenza, human metapneumovirus, and a combination thereof. In some embodiments, the one or more second viruses consists of respiratory syncytial virus (RSV), Influenza, human metapneumovirus, and a combination thereof. In some embodiments, the composition comprises 1, 2, 3, or 4 first antigens. In some embodiments, the first antigen is selected from the group consisting of PIV-3 antigen M, PIV-3 antigen HN, PIV-3 antigen N, PIV-3 antigen F, and combinations thereof. In some embodiments, the 4 first antigens are as follows: PIV-3 antigen M, PIV-3 antigen HN, PIV-3 antigen N, and PIV-3 antigen F. In some embodiments, the composition comprises two or three second viruses. In some embodiments, the composition comprises three second viruses. In some embodiments, the three second viruses are influenza, RSV, and hMPV. In some embodiments, the composition comprises at least two second antigens per each second virus. In some embodiments, the composition comprises 1, 2, 3, 4, 5, 6, 7, or 8 second antigens. In some embodiments, the second antigen is selected from the group consisting of influenza antigen NP1, influenza antigen MP1, RSV antigen N, RSV antigen F, hMPV antigen M, hMPV antigen M2-1, hMPV antigen F, hMPV antigen N, and combinations thereof. In some embodiments, the second antigen comprises influenza antigen NP1, influenza antigen MP1, or both. In some embodiments, the second antigen comprises RSV antigen N, RSV antigen F, or both. In some embodiments, the second antigen comprises hMPV antigen M, hMPV antigen M2-1, hMPV antigen F, hMPV antigen N, and combinations thereof. In some embodiments, the second antigen comprises each of influenza antigen NP1, influenza antigen MP1, RSV antigen N, RSV antigen F, hMPV antigen M, hMPV antigen M2-1, hMPV antigen F, hMPV antigen N. In some embodiments, the plurality of antigens comprise PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens consist of, or consist essentially of, PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N. In some embodiments, the VSTs are cultured ex vivo in the presence of both IL-7 and IL-4. In some embodiments, the multivirus VSTs have expanded sufficiently within 9-18 days of culture such that they are ready for administration to a subject. In some embodiments, the VSTs exhibit one or more properties selected from (a) negligible alloreactivity; (b) less activation induced cell death of antigen-specific T cells harvested from a subject than corresponding antigen-specific T cells harvested from the same subject, but not cultured in the presence of both IL-7 and IL-4; and (c) viability of greater than 70%. In some embodiments, the composition is negative for bacteria and fungi for at least 7 days in culture; exhibit less than 5 EU/ml of endotoxin, and are negative for mycoplasma. In some embodiments, the pepmixes were chemically synthesized and are, optionally >90% pure. In some embodiments, the VSTs are Th1 polarized. In some embodiments, the VSTs are able to lyse viral antigen-expressing target cells. In some embodiments, the VSTs do not significantly lyse non-infected autologous or allogenic target cells.

The present disclosure also provides a pharmaceutical composition comprising any one of the compositions disclosed herein, formulated for intravenous delivery, wherein the composition is negative for bacteria and fungi for at least 7 days in culture; exhibit less than 5 EU/ml of endotoxin, and are negative for mycoplasma. For example, in some embodiments, the present disclosure provides a pharmaceutical composition comprising a polyclonal population of virus specific T-lymphocytes (VSTs) that recognize a plurality of viral antigens, wherein the plurality of viral antigens comprise at least one first antigen from PIV and at least one second antigen from one or more second viruses. In some embodiments, the second virus comprises respiratory syncytial virus (RSV), Influenza, and human metapneumovirus. In some embodiments, the VSTs are generated by contacting peripheral blood mononuclear cells (PBMCs) with a plurality of pepmix libraries, each pepmix library containing a plurality of overlapping peptides spanning at least a portion of a viral antigen, wherein at least one of the plurality of pepmix libraries spans a first antigen from PIV-3 and wherein at least one additional pepmix library of the plurality of pepmix libraries spans each second antigen, wherein the pharmaceutical composition is formulated for intravenous delivery, wherein the composition is negative for bacteria and fungi for at least 7 days in culture; exhibit less than 5 EU/ml of endotoxin, and are negative for mycoplasma.

The present disclosure also provides a method of lysing a target cell with any one or more of the compositions or pharmaceutical compositions disclosed herein. For example, in some embodiments, the present disclosure provides a method of lysing a target cell comprising contacting the target cell with a polyclonal population of virus specific T-lymphocytes (VSTs) that recognizes a plurality of viral antigens, wherein the plurality of viral antigens comprise at least one first antigen from PIV and at least one second antigen from one or more second viruses. In some embodiments, the second virus comprises respiratory syncytial virus (RSV), Influenza, and human metapneumovirus. In some embodiments, the VSTs are generated by contacting peripheral blood mononuclear cells (PBMCs) with a plurality of pepmix libraries, each pepmix library containing a plurality of overlapping peptides spanning at least a portion of a viral antigen, wherein at least one of the plurality of pepmix libraries spans a first antigen from PIV-3 and wherein at least one additional pepmix library of the plurality of pepmix libraries spans each second antigen. In some embodiments, the contacting occurs in vivo in a subject. In some embodiments, the contacting occurs in vivo via administration of the VSTs to a subject.

The present disclosure also provides a method of treating or preventing a viral infection comprising administering to a subject in need thereof any one or more of the compositions or pharmaceutical compositions disclosed herein. For example, in some embodiments, the present disclosure provides a method of treating or preventing a viral infection comprising administering to a subject in need thereof a polyclonal population of virus specific T-lymphocytes (VSTs) that recognize a plurality of viral antigens, wherein the plurality of viral antigens comprise at least one first antigen from PIV and at least one second antigen from one or more second viruses. In some embodiments, the second virus comprises respiratory syncytial virus (RSV), Influenza, and human metapneumovirus. In some embodiments, the VSTs are generated by contacting peripheral blood mononuclear cells (PBMCs) with a plurality of pepmix libraries, each pepmix library containing a plurality of overlapping peptides spanning at least a portion of a viral antigen, wherein at least one of the plurality of pepmix libraries spans a first antigen from PIV-3 and wherein at least one additional pepmix library of the plurality of pepmix libraries spans each second antigen. In some embodiments, between 5×10⁶ and 5×10⁷ VSTs/m² administered to the subject. In some embodiments, the subject is administered the VSTs in multiple doses. In one embodiment, the subject is administered the VSTs and then the subject's viral load is monitored and if the viral load increases the subject is administered a second dose of the VSTs. In some embodiments, the subject is immunocompromised. In some embodiments, the subject has acute myeloid leukemia, acute lymphoblastic leukemia, or chronic granulomatous disease. In some embodiments, the subject, prior to receiving the VSTs, received: (a) a matched related donor transplant with reduced intensity conditioning; (b) a matched unrelated donor transplant with myeloablative conditioning; (c) a haplo-identical transplant with reduced intensity conditioning; or (d) a matched related donor transplant with myeloablative conditioning. In some embodiments, the subject (a) has received a solid organ transplantation; (b) has received chemotherapy; (c) has an HIV infection; (d) has a genetic immunodeficiency; and/or (e) has received an allogeneic stem cell transplant. In some embodiments, the composition is administered to the subject a plurality of times. In some embodiments, the administration of the composition effectively treats or prevents a viral infection in the subject, wherein the viral infection is selected from the group consisting of parainfluenza virus type 3, respiratory syncytial virus, Influenza, human metapneumovirus, and a combination thereof. In some embodiments, the subject is a human.

The present disclosure also provides a composition comprising a polyclonal population of VSTs that recognize a plurality of viral antigens, wherein the plurality of viral antigens comprises at least one antigen selected from parainfluenza virus type 3 (PIV-3), respiratory syncytial virus, Influenza, human metapneumovirus, and a combination thereof. In some embodiments, VSTs recognize a plurality of viral antigens, wherein the plurality of viral antigens comprises at least one antigen from each of parainfluenza virus type 3, respiratory syncytial virus, Influenza, and human metapneumovirus. In some embodiments, the VSTs recognize a plurality of viral antigens, wherein the plurality of viral antigens comprise at least two antigens from each of parainfluenza virus type 3, respiratory syncytial virus, Influenza, and human metapneumovirus. In some embodiments, the plurality of antigens comprise, consist of, or consist essentially of, PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N. In some embodiments, the composition is a pharmaceutical composition formulated for intravenous delivery. In some embodiments, the composition is negative for bacteria and fungi for at least 7 days in culture; exhibit less than 5 EU/ml of endotoxin, and are negative for mycoplasma.

The present disclosure also provides a method of lysing a target cell comprising contacting the target cell with a composition comprising a polyclonal population of VSTs that recognize a plurality of viral antigens, wherein the plurality of viral antigens comprise at least one antigen selected from parainfluenza virus type 3 (PIV-3), respiratory syncytial virus, Influenza, and human metapneumovirus. In some embodiments, the composition is a pharmaceutical composition. In some embodiments, the contacting occurs in vivo in a subject. In some embodiments, the contacting occurs in vivo via administration of the VSTs to a subject.

The present disclosure also provides a method of treating or preventing a viral infection comprising administering to a subject in need thereof cell a composition comprising a polyclonal population of VSTs that recognize a plurality of viral antigens, wherein the plurality of viral antigens comprise at least one antigen selected from parainfluenza virus type 3 (PIV-3), respiratory syncytial virus, Influenza, and human metapneumovirus. In some embodiments, the composition is a pharmaceutical composition. In some embodiments, the composition is administered to the subject a plurality of times. In some embodiments, the administration of the composition effectively treats or prevents a viral infection in the subject, wherein the viral infection is selected from the group consisting of parainfluenza virus type 3, respiratory syncytial virus, Influenza, and human metapneumovirus. In some embodiments, the subject is a human.

### BRIEF DESCRIPTION OF THE DRAWINGS

FIG. 1: Generation of polyclonal multi-respiratory virus-specific T cells (multi-R-VSTs) from healthy donors. FIG. 1A shows a schematic of the multi-R-VST generation protocol. FIG. 1B shows the fold expansion achieved over a 10-day period based on cell counting using trypan blue exclusion (n=12). Manufacturing runs may be anywhere from about 10-18 days. FIG. 1C and FIG. 1D show the phenotype of the expanded cells (mean±SEM, n=12). FIG. 1E shows minimal detection of Tregs (CD4+CD25+FoxP3+) within the expanded CD4+ T cell populations (mean±SEM, n=8).

FIG. 2: Specificity and enrichment of multi-R-VSTs. FIG. 2A shows the specificity of virus-reactive T cells within the expanded T cell lines following exposure to individual stimulating antigens from each of the target viruses. Data is presented as mean±SEM SFC/2×10⁵ (n=12). FIG. 2B shows fold enrichment of specificity (PBMC vs multi-R-VST; n=12). FIG. 2C shows IFNγ production, as assessed by ICS from CD4 helper (top) and CD8 cytotoxic T cells (bottom) after viral stimulation in 1 representative donor (dot plots were gated on CD3+ cells) while FIG. 2D shows summary results for 9 donors screened (mean±SEM). FIG. 2E shows the number of donor-derived VST lines responding to individual stimulating antigens. FIG. 2F shows specificity of virus-reactive T cells within expanded T cell lines following exposure to titrated concentrations of pooled stimulating antigens from each of the target viruses. Data is presented as mean±SEM SFC/2×10⁵ (n=7). FIG. 2G shows the frequency of CARV-specific T cells in the peripheral blood of healthy donors following exposure to individual stimulating antigens from each of the target viruses. Data is presented as mean±SEM SFC/5×10⁵ (n=12).

FIG. 3: Multi-R-VSTs are polyclonal and polyfunctional. FIG. 3A shows dual IFNγ and TNFα production from CD3+ T cells as assessed by ICS in 1 representative donor, while FIG. 3B shows summary results from 9 donors screened (mean±SEM). FIG. 3C shows the cytokine profile of multi-R-VSTs as measured by multiplex bead array, while FIG. 3D assesses the production of Granzyme B by ELIspot assay. Results are reported as SFC/2×10⁵ input VSTs (mean±SEM, n=9).

FIG. 4: Multi-R-VSTs are reactive against virus-infected targets. FIG. 4A shows the cytolytic potential of multi-R-VSTs evaluated by standard 4-hour Cr51 release assay using autologous pepmix-pulsed PHA blasts as targets (E:T 40:1; n=8) with unloaded PHA blasts as a control. Results are presented as percentage of specific lysis (mean±SEM). FIG. 4B demonstrates that multi-R-VSTs show negligible activity against either non-infected autologous or allogeneic PHA blasts, as assessed by Cr51 release assay. FIG. 4C shows cytotoxic activity of multi-R-VSTs evaluated by standard 4-hour Cr51 release assay using autologous pepmix-pulsed PHA blasts as targets (E:T 40:1, 20:1, 10:1, 5:1) with unloaded PHA blasts as a control. Results are presented as percentage of specific lysis (mean±SEM, n=8).

FIG. 5: Detection of respiratory syncytial virus (RSV)- and human metapneumovirus (hMPV)-specific T cells in the peripheral blood of HSCT recipients. PBMCs isolated from 2 HSCT recipients with 3 infections were tested for specificity against the infecting viruses, using IFNγ ELIspot as a readout. FIG. 5A and FIG. 5B show results from 2 patients with RSV-associated URTIs which were controlled, coincident with a detectable rise in endogenous RSV-specific T cells while FIG. 5C shows clearance of an hMPV-LRTI with expansion of endogenous hMPV-specific T cells. ALC: absolute lymphocyte count.

FIG. 6: Detection of RSV- and parainfluenza (PIV-3)-specific T cells in the peripheral blood of HSCT recipients. PBMCs isolated from 3 HSCT recipients with 3 infections were tested for specificity against the infecting viruses, using IFNγ ELIspot as a readout. FIG. 6A and FIG. 6B show results from 2 patients with RSV- and PIV-associated URTIs and LRTIs which were controlled, coincident with a detectable rise in endogenous virus-specific T cells. FIG. 6C shows results from a patient with an ongoing PIV-related severe URTI who failed to mount a T cell response against the virus. ALC: absolute lymphocyte count.

FIG. 7. Structure of the RSV genome and morphology.

FIG. 8. Schematic of the RSV-VST generation protocol.

FIG. 9. Characterization of RSV-VSTs. FIG. 9A shows the fold expansion achieved over a 10-day period based on cell counting using trypan blue exclusion. FIG. 9B and FIG. 9C show the phenotype of the expanded cells.

FIG. 10. RSV-VSTs are polyfunctional. FIG. 10A shows IFNγ production from CD3+ T cells as assessed by EliSpot assay. FIG. 10B shows production of Granzyme B by ELIspot assay. Results are reported as SFC/2×10⁵ input VSTs (mean±SEM, n=9).

FIG. 11. Cytokine profile of RSV-VSTs as measured by multiplex bead array.

### DETAILED DESCRIPTION

### Definitions

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The term “about” when immediately preceding a numerical value means±0% to 10% of the numerical value, ±0% to 10%, ±0% to 9%, ±0% to 8%, ±0% to 7%, ±0% to 6%, ±0% to 5%, ±0% to 4%, ±0% to 3%, ±0% to 2%, ±0% to 1%, ±0% to less than 1%, or any other value or range of values therein. For example, “about 40” means±0% to 10% of 40 (i.e., from 36 to 44).

The use of the term “or” in the claims is used to mean “and/or” unless explicitly indicated to refer to alternatives only or the alternatives are mutually exclusive, although the disclosure supports a definition that refers to only alternatives and “and/or.”

The term “viral antigen” as used herein refers to an antigen that is proteinaceous in nature. In specific embodiments, a viral antigen is a coat protein. Specific examples of viral antigens include antigens from at least a virus selected from EBV, CMV, AdV, BK, JC virus, HHV6, RSV, Influenza, Parainfluenza, Bocavirus, Coronavirus, Rhinovirus, LCMV, Mumps, Measles, hMPV, Parvovirus B, Rotavirus, Merkel cell virus, herpes simplex virus, HPV, HIV, HTLV1, HHV8, Hepatitis C, Hepatitis B, HTLV1, Herpes simplex virus, West Nile Virus, zika virus, and Ebola.

The term “antigen-specific T cell lines” or “virus-specific T cells” or “virus-specific T cell lines” are used interchangeably herein to refer to polyclonal T cell lines that have specificity and potency against a virus or viruses of interest. As described herein, a viral antigen or several viral antigens are presented to native T cells in peripheral blood mononuclear cells and the native CD4+ and CD8+ T cell populations expand in response to said viral antigen(s). For example, an antigen-specific T cell line or a virus-specific T cell for EBV can recognize EBV, thereby expanding the T cells specific for EBV. In another example, an antigen-specific T cell line or a virus-specific T cell for adenovirus and BK can recognize both AdV and BK, thereby expanding the T cells specific for adenovirus and BK.

As used herein, the terms “patient” or “subject” or “individual” as used interchangeably herein to refer to any mammal, including humans, domestic and farm animals, and zoo, sports, and pet animals, such as dogs, horses, cats, and agricultural use animals including cattle, sheep, pigs, and goats. One particular mammal is a human, including adults, children, and the elderly. A subject may also be a pet animal, including dogs, cats and horses. Examples of agricultural animals include pigs, cattle, sheep, and goats.

The terms “treat”, “treating”, “treatment” and the like, as used herein, unless otherwise indicated, refers to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the disease, condition, or disorder. In some instances, treatment is curative or ameliorating.

The terms “administering”, “administer”, “administration” and the like, as used herein, refer to any mode of transferring, delivering, introducing, or transporting a therapeutic agent to a subject in need of treatment with such an agent. Such modes include, but are not limited to, intraocular, oral, topical, intravenous, intraperitoneal, intramuscular, intradermal, intranasal, and subcutaneous administration.

As used herein, the terms “comprise,” “comprising,” “includes,” “including,” “has,” “having,” “contains,” “containing,” “characterized by,” or any other variation thereof, are intended to encompass a non-exclusive inclusion, subject to any limitation explicitly indicated otherwise, of the recited components. For example, a composition and/or method that “comprises” a list of elements (e.g., components or features or steps) is not necessarily limited to only those elements (or components or features or steps), but may include other elements (or components or features or steps) not expressly listed or inherent to the composition and/or method.

As used herein, the phrases “consists of” and “consisting of” exclude any element, step, or component not specified. For example, “consist of” or “consisting of” used in a claim would limit the claim to the components, materials or steps specifically recited in the claim except for impurities ordinarily associated with therewith (i.e., impurities within a given component). When the phrase “consist of” or “consisting of” appears in a clause of the body of a claim, rather than immediately following the preamble, the phrase “consist of” or “consisting of” limits only the elements (or components or steps) set forth in that clause; other elements (or components) are not excluded from the claim as a whole.

Other objects, feature and advantages of the present invention will become apparent from the following detailed description. It should be understood, however, that the detailed description and specific examples, while indicating specific embodiments of the invention, are given by way of illustration only, since various changes and modifications within the spirit and scope of the invention will become apparent to those skilled in the art from this detailed description.

The following discussion is directed to various embodiments of the invention. The term “invention” is not intended to refer to any particular embodiment or otherwise limit the scope of the disclosure. Although one or more of these embodiments may be employed, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

### Overview

In various embodiments, the present disclosure provides compositions and methods for treating or preventing viral infections (e.g., respiratory viral infections) and associated diseases. The present disclosure relates to the prevention or treatment of such infections by the administration of ex vivo expanded, non-genetically modified, virus-specific T cells (VSTs) to control viral infections and eliminate symptoms. Without wishing to be bound by any theories, VSTs recognize and kill virus-infected cells via their native T cell receptor (TCR), which binds to major histocompatibility complex (MHC) molecules expressed on target cells that present virus-derived peptides.

Respiratory viral infections due to community-acquired respiratory viruses (CARVs) including respiratory syncytial virus (RSV), influenza, parainfluenza virus (PIV) and human metapneumovirus (hMPV) are detected in up to 40% of allogeneic hematopoietic stem cell transplant (allo-HSCT) recipients, in whom they may cause severe disease such as bronchiolitis and pneumonia that can be fatal. RSV induced bronchiolitis is the most common reason for hospital admission in children less than 1 year, while the Center for Disease Control (CDC) estimates that, annually, Influenza accounts for up to 35.6 million illnesses worldwide, between 140,000 and 710,000 hospitalizations, annual costs of approximately $87.1 billion in disease management in the US alone and between 12,000 and 56,000 deaths.

Thus, CARVs are a leading cause of morbidity and mortality worldwide, with individuals whose immune systems are naïve (e.g. young children) or compromised being most vulnerable. For example, in allogeneic hematopoietic stem cell transplant (HSCT) recipients, the incidence of CARV-related respiratory viral diseases is as high as 40%(5). While most patients initially present with rhinorrhea, cough and fever, in approximately 50% of cases infections progress to the lower respiratory tract and are characterized by severe symptoms including pneumonia and bronchiolitis and mortality rates of 23-50%(6-9). There are neither approved preventative vaccines nor antiviral drugs for hMPV(10) and PIV(11) and for Influenza the preventative vaccine is not indicated unless patients are at least 6 months post-HSCT(12). Aerosolized ribavirin (RBV) is FDA-approved for the treatment of RSV, but it is extremely costly (5-day course=$149,756) and logistically difficult to administer, requiring a specialized nebulization device that connects to an aerosol tent surrounding the patient(13-16). Thus, the lack of approved antiviral agents for many clinically problematic CARVs and high cost and complexity of administering aerosolized RBV underscores the need for alternative treatment strategies.

Other respiratory viruses including adenovirus (AdV), Rhinovirus and the coronaviruses strains SARS-CoV, SARS-CoV-2, MERS-CoV, as well as the endemic CoVs that afflict both immunocompetent and immunocompromised patients. These can cause severe symptoms, especially in immunocompromised individuals, and the SARS-CoV2 pandemic of 2020 has clearly exposed how ill-prepared humans are to treat and prevent this infection and associated disease. This horrible pandemic has already resulted in thousands of deaths worldwide, the collapse of healthcare systems, and a global economic meltdown not seen in decades. Thus, it is clear there is an urgent need for new therapies to treat these viruses. The present disclosure provides such a therapy.

In some embodiments, the present disclosure provides VSTs produced from peripheral blood mononuclear cells (PBMCs) procured from healthy, pre-screened, seropositive donors, which are available as a partially HLA-matched “off-the-shelf” product. Accordingly, the present disclosure provides VST products comprising VST with specificity for one or more viruses and methods of using such VSTs for treating or preventing viral infections.

In some embodiments, the VSTs described herein respond to (or “are specific for”) one or more virus (e.g., one or more respiratory virus) or more specifically one or more antigens expressed by the virus. In some embodiments, the VSTs described herein respond to only one virus. For example, in one embodiment, the present disclosure provides a polyclonal population of VSTs with specificity for one or more RSV antigens. In some instances, such RSV-specific VSTs comprise T cells with specificity for a plurality of RSV antigens. In some embodiments, the present disclosure also provides methods of treating an RSV infection in a subject by administering such RSV-specific VSTs. In some embodiments, the present disclosure also provides methods of preventing an RSV infection in a subject by administering such RSV-specific VSTs. Such practices may be applied to any single virus other than RSV.

In some embodiments, the VSTs described herein respond to more than one virus (e.g., any one or more viruses disclosed herein). In particular embodiments, the present disclosure provides multi-respiratory virus specific T cells (multi-R-VSTs) that respond to more than one respiratory virus (e.g., any one or more of the respiratory viruses disclosed herein). In certain aspects the multi-R-VSTs have specificity to one or more respiratory virus antigens expressed by a virus selected from Influenza, RSV, hMPV, PIV, and a combination thereof. In particular embodiments, the multi-R-VSTs have specificity to antigens expressed by each of Influenza, RSV, hMPV, and PIV. In certain aspects the multi-R-VSTs have specificity to one or more respiratory virus antigens expressed by a virus selected from Influenza, RSV, hMPV, PIV3, and a combination thereof. In particular embodiments, the multi-R-VSTs have specificity to antigens expressed by each of Influenza, RSV, hMPV, and PIV3. In some embodiments, the influenza antigen is influenza A antigen NP1. In some embodiments, the influenza antigen is influenza A antigen MP1. In some embodiments, the influenza antigen is a combination of NP1 and MP1. In some embodiments, the RSV antigen is RSV N. In some embodiments, the RSV antigen is RSV F. In some embodiments, the RSV antigen is a combination of RSV N and F. In some embodiments, the hMPV antigen is F. In some embodiments, the hMPV antigen is N. In some embodiments, the hMPV antigen is M2-1. In some embodiments, the hMPV antigen is M. In some embodiments, the hMPV antigen is a combination of F, N, M2-1, and M. In some embodiments, the PIV antigen is M. In some embodiments, the PIV antigen is HN. In some embodiments, the PIV antigen is N. In some embodiments, the PIV antigen is F. In some embodiments, the PIV antigen is a combination of M, HN, N, and F. In some embodiments, the present disclosure also provides methods of treating a PIV, influenza, RSV, and/or hMPV infection in a subject by administering such multi-R-VSTs to the subject. In some embodiments, the present disclosure also provides methods of preventing a PIV, influenza, RSV, and/or hMPV infection in a subject by administering such multi-R-VSTs to the subject. In some embodiments, the PIV3 antigen is M. In some embodiments, the PIV3 antigen is HN. In some embodiments, the PIV3 antigen is N. In some embodiments, the PIV3 antigen is F. In some embodiments, the PIV3 antigen is a combination of M, HN, N, and F. In some embodiments, the present disclosure also provides methods of treating a PIV3, influenza, RSV, and/or hMPV infection in a subject by administering such multi-R-VSTs to the subject. In some embodiments, the present disclosure also provides methods of preventing a PIV3, influenza, RSV, and/or hMPV infection in a subject by administering such multi-R-VSTs to the subject. Such practices may be applied to any multiple viruses.

In one particular embodiment, the present disclosure provides a composition comprising a polyclonal population of multi-R-VSTs with specificity for each of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. The polyclonal population may include both CD4+ and CD8+VSTs. The polyclonal population may be administered to a subject. The subject may have a PIV, influenza, RSV, and/or hMPV infection. A method of treating a PIV, influenza, RSV, and/or hMPV infection in a subject may comprise administering to the subject the polyclonal population of multi-R-VSTs. A method of preventing a PIV, influenza, RSV, and/or hMPV infection in a subject may comprise administering to the subject the polyclonal population of multi-R-VSTs.

In one particular embodiment, the present disclosure provides a composition comprising a polyclonal population of multi-R-VSTs with specificity for each of PIV3 antigen M, PIV3 antigen HN, PIV3 antigen N, PIV3 antigen F, influenza antigen NP1, influenza antigen MP1, RSV antigen N, RSV antigen F, hMPV antigen M, hMPV antigen M2-1, hMPV antigen F, and hMPV antigen N. The polyclonal population may include both CD4+ and CD8+VSTs. The polyclonal population may be administered to a subject. The subject may have a PIV3, influenza, RSV, and/or hMPV infection. A method of treating a PIV3, influenza, RSV, and/or hMPV infection in a subject may comprise administering to the subject the polyclonal population of multi-R-VSTs. A method of preventing a PIV3, influenza, RSV, and/or hMPV infection in a subject may comprise administering to the subject the polyclonal population of multi-R-VSTs.

In some embodiments, the present disclosure provides a composition comprising a polyclonal population of VSTs that recognize a plurality of viral antigens, wherein the plurality of viral antigens comprises at least one first antigen from PIV and at least one second antigen from one or more additional virus. In some particular embodiments, the present disclosure provides a composition comprising a polyclonal population of VSTs that recognize a plurality of viral antigens, wherein the plurality of viral antigens comprises at least one first antigen from PIV3 and at least one second antigen from one or more additional viruses. The additional virus may comprise influenza, RSV, hMPV, AdV, coronavirus, or a combination thereof. The VSTs may recognize an additional antigen expressed by the one or more additional viruses, wherein the additional antigen may comprise one or more or all of the group consisting of PIV antigen M (e.g., PIV3 antigen M), PIV antigen HN (e.g., PIV3 antigen HN), PIV antigen N (e.g., PIV3 antigen N), PIV antigen F (e.g., PIV3 antigen F), influenza antigen NP1, influenza antigen MP1, RSV antigen N, RSV antigen F, hMPV antigen M, hMPV antigen M2-1, hMPV antigen F, hMPV antigen N, and AdV antigen Hexon, AdV antigen Penton and combinations thereof. The additional antigen may in some embodiments comprise one or more coronavirus antigens. For example, the additional antigen may comprise one or more coronavirus (e.g., SARS-CoV or SARS-CoV2) antigens. In some embodiments, the coronavirus antigen comprises one or more SARS-CoV2 antigen selected from the group consisting of nspl; nsp3; nsp4; nsp5; nsp6; nsp10; nsp12; nsp13; nsp14; nsp15; nsp16; Spike (S); Envelope protein (E); Matrix protein (M); Nucleocapsid protein (N). In some embodiments, the SARS-CoV2 antigen further comprises one or more antigen selected from the group consisting of SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14).

The additional antigen may in some embodiments additionally or alternatively be from a virus selected from EBV, CMV, AdV, BK, JC virus, HHV6, RSV, Influenza, Parainfluenza, Bocavirus, Rhinovirus, Coronavirus, LCMV, Mumps, Measles, human Metapneumovirus, Parvovirus B, Rotavirus, Merkel cell virus, Herpes simplex virus, HPV, HIV, HTLV1, HHV8, Hepatitis C, Hepatitis B, HTLV1, and West Nile Virus, zika virus, Ebola. In some embodiments, the EBV antigens are from LMP2, EBNA1, BZLF1, and a combination thereof. In some embodiments, the CMV antigens are from 1E1, pp65, and a combination thereof. In some embodiments, the adenovirus antigens are from Hexon, Penton, and a combination thereof. In some embodiments, the BK virus antigens are from VP1, large T, and a combination thereof. In some embodiments, the HHV6 antigens are from U90, U11, U14, and a combination thereof.

In some embodiments, at least one pepmix covers an antigen (or part of an antigen) from RSV, Influenza, PIV, or hMPV. In some embodiments, at least one pepmix covers an antigen (or part of an antigen) from RSV, Influenza, PIV, hMPV, a coronavirus (e.g., SARS-CoV or SARS-CoV2), or a combination thereof. In some embodiments, at least one pepmix covers an antigen (or part of an antigen) from RSV, Influenza, PIV3, hMPV, or a combination thereof. In some embodiments, at least one pepmix covers an antigen (or part of an antigen) from RSV, Influenza, PIV3, hMPV, a coronavirus (e.g., SARS-CoV or SARS-CoV2), or a combination thereof.

In some embodiments, the first antigen is a PIV antigen. For example, in some embodiments, the first antigen can be PIV antigen M. In some embodiments, the first antigen can be PIV antigen HN. In some embodiments, the first antigen can be PIV antigen N. In some embodiments, the first antigen can be PIV antigen F. In some embodiments, the first antigen can be any combinations of PIV antigen M, PIV antigen HN, PIV antigen N, and PIV antigen F. In some embodiments, the composition can comprise 1 first antigen. In some embodiments, the composition can comprise 2 first antigens. In some embodiments, the composition can comprise 3 first antigens. In some embodiments, the composition can comprise 4 first antigens. In some embodiments, the 4 first antigens can comprise PIV antigen M, PIV antigen HN, PIV antigen N, and PIV antigen F.

In some embodiments, the first antigen is a PIV3 antigen. For example, in some embodiments, the first antigen can be PIV3 antigen M. In some embodiments, the first antigen can be PIV3 antigen HN. In some embodiments, the first antigen can be PIV3 antigen N. In some embodiments, the first antigen can be PIV3 antigen F. In some embodiments, the first antigen can be any combinations of PIV3 antigen M, PIV3 antigen HN, PIV3 antigen N, and PIV3 antigen F. In some embodiments, the composition can comprise 1 first antigen. In some embodiments, the composition can comprise 2 first antigens. In some embodiments, the composition can comprise 3 first antigens. In some embodiments, the composition can comprise 4 first antigens. In some embodiments, the 4 first antigens can comprise PIV3 antigen M, PIV3 antigen HN, PIV3 antigen N, and PIV3 antigen F.

In some embodiments, the one or more second viruses can be RSV. In some embodiments, the one or more second viruses can be Influenza. In some embodiments, the one or more second viruses can be hMPV. In some embodiments, the one or more second viruses can comprises RSV, Influenza, and hMPV. In some embodiments, the one or more second viruses can consist of RSV, Influenza, and hMPV. In some embodiments, the one or more second viruses can be selected from any suitable viruses as described herein.

In some embodiments, the composition can comprise two or three second viruses. In some embodiments, the composition can comprise three second viruses. In some embodiments, the three second viruses can comprise influenza, RSV, and hMPV. In some embodiments, the composition comprise at least two second antigens per each second virus. In some embodiments, the composition comprises 1 second antigen. In some embodiments, the composition comprises 2 second antigens. In some embodiments, the composition comprises 3 second antigens. In some embodiments, the composition comprises 4 second antigens. In some embodiments, the composition comprises 5 second antigens. In some embodiments, the composition comprises 6 second antigens. In some embodiments, the composition comprises 7 second antigens. In some embodiments, the composition comprises 8 second antigens. In some embodiments, the composition comprises 9 second antigens. In some embodiments, the composition comprises 10 second antigens. In some embodiments, the composition comprises 11 second antigens. In some embodiments, the composition comprises 12 second antigens. In some embodiments, the composition comprises any numbers of second antigens that would be suitable for the compositions as described herein.

In some embodiments, the second antigen can be influenza antigen NP1. In some embodiments, the second antigen can be influenza antigen MP1. In some embodiments, the second antigen can be RSV antigen N. In some embodiments, the second antigen can be RSV antigen F. In some embodiments, the second antigen can be hMPV antigen M. In some embodiments, the second antigen can be hMPV antigen M2-1. In some embodiments, the second antigen can be hMPV antigen F. In some embodiments, the second antigen can be hMPV antigen N. In some embodiments, the second antigen can be any combinations of influenza antigen NP1, influenza antigen MP1, RSV antigen N, RSV antigen F, hMPV antigen M, hMPV antigen M2-1, hMPV antigen F, and hMPV antigen N.

In some embodiments, the second antigen comprises influenza antigen NP1. In some embodiments, the second antigen comprises influenza antigen MP1. In some embodiments, In some embodiments, the second antigen comprises both influenza antigen NP1 and influenza antigen MP1. In some embodiments, the second antigen comprises RSV antigen N. In some embodiments, the second antigen comprises RSV antigen F. In some embodiments, the second antigen comprises both RSV antigen N and RSV antigen F.

In some embodiments, the second antigen comprises hMPV antigen M. In some embodiments, the second antigen comprises hMPV antigen M2-1. In some embodiments, the second antigen comprises hMPV antigen F. In some embodiments, the second antigen comprises hMPV antigen N. In some embodiments, the second antigen comprises combinations of hMPV antigen M, hMPV antigen M2-1, hMPV antigen F, and hMPV antigen N.

In some embodiments, the second antigen comprises each of influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens comprise PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens consist of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens consist essentially of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. In some embodiments, the second antigen can comprise any suitable antigens for the compositions as described herein.

In some embodiments, the second antigen comprises each of influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens comprise PIV3 antigen M, PIV3 antigen HN, PIV3 antigen N, PIV3 antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens consist of PIV3 antigen M, PIV3 antigen HN, PIV3 antigen N, PIV3 antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens consist essentially of PIV3 antigen M, PIV3 antigen HN, PIV3 antigen N, PIV3 antigen F, influenza antigen NP1, influenza antigen MP1, RSV antigen N, RSV antigen F, hMPV antigen M, hMPV antigen M2-1, hMPV antigen F, and hMPV antigen N. In some embodiments, the second antigen can comprise any suitable antigens for the compositions as described herein.

In some embodiments, the VSTs in the compositions disclosed herein are generated by contacting PBMCs with a plurality of pepmix libraries. In some embodiments, each pepmix library contains a plurality of overlapping peptides spanning at least a portion of a viral antigen. In some embodiments, at least one of the plurality of pepmix libraries spans a first antigen from PIV. In some embodiments, at least one of the plurality of pepmix libraries spans a first antigen from PIV3. In some embodiments, at least one additional pepmix library of the plurality of pepmix libraries spans each second antigen.

In some embodiments, the VSTs disclosed herein are generated by contacting T cells with antigen presenting cells (APCs) such as dendritic cells (DCs) nucleofected with at least one DNA plasmid. In some embodiments, the DNA plasmid can encode at least a portion of one antigen. In some embodiments, the DNA plasmid can encode a PIV antigen (e.g., a PIV3 antigen). In some embodiments, the at least one DNA plasmid encodes each second antigen. In some embodiments, the plasmid encodes at least one PIV antigen and at least one of the second antigens. In some embodiments, the compositions as described herein comprise CD4+T-lymphocytes and CD8+T-lymphocytes. In some embodiments, the compositions comprise VSTs expressing αβ T cell receptors. In some embodiments, the compositions comprise MHC-restricted VSTs.

In some embodiments, the present disclosure provides multi-respiratory virus specific T cells (multi-R-VSTs) with specificity to one or more respiratory viruses selected from Influenza, RSV, hMPV, PIV, and one or more additional viruses. The PIV antigen may be from PIV3. For example, in some instances, the additional virus comprises a coronavirus. The coronavirus may be an alpha coronavirus. For example, in particular embodiments, the alpha coronavirus is selected from HCoV-E229, HCoV-NL63, and a combination thereof. In particular embodiments, the alpha coronavirus comprises each of HCoV-E229 and HCoV-NL63. The coronavirus may be a beta coronavirus. For example, in particular embodiments, the beta coronavirus is selected from SARS-CoV, SARS-CoV2, MERS-CoV, HCoV-HKU1, HCoV-0C43, and a combination thereof. In particular embodiments, the beta coronavirus comprises each of SARS-CoV, SARS-CoV2, MERS-CoV, HCoV-HKU1, HCoV-0C43, and a combination thereof. In some instances the additional virus comprises an adenovirus. In some instances, the additional virus is selected from the group consisting of EBV, CMV, AdV, BK, JC virus, HHV6, Bocavirus, Rhinovirus, Coronavirus, LCMV, Mumps, Measles, Parvovirus B, Rotavirus, Merkel cell virus, herpes simplex virus, HPV, HIV, HTLV1, HHV8, Hepatitis C, Hepatitis B, HTLV1, West Nile Virus, zika virus, Ebola, and a combination thereof.

In one embodiment, the present disclosure provides multi-R-VST with specificity to Influenza, RSV, hMPV, PIV, and a coronavirus (e.g., SARS-CoV2). In one embodiment, the present disclosure provides multi-R-VST with specificity to Influenza, RSV, hMPV, PIV, one or more AdV, and a coronavirus (e.g., SARS-CoV or SARS-CoV2). In one embodiment, the present disclosure provides multi-R-VST with specificity to Influenza, RSV, hMPV, PIV3, and a coronavirus (e.g. SARS-CoV2). In one embodiment, the present disclosure provides multi-R-VST with specificity to Influenza, RSV, hMPV, PIV3, one or more AdV, and a coronavirus (e.g., SARS-CoV or SARS-CoV2).

In some embodiments, the VSTs can be cultured ex vivo in the presence of both IL-7 and IL-4. In some embodiments, the multivirus VSTs have expanded sufficiently within 9 days, 10 days, 11 days, 12 days, 13 days, 14 days, 15 days, 16 days, 17 days, 18 days, 19 days, 20 days inclusive of all ranges and subranges therebetween, of culture such that they are ready for administration to a patient. Typical manufacturing runs (culturing/expanding in the above conditions) are for 10-18 days, more typically 14-16 days. In some embodiments, the multivirus VSTs have expanded sufficiently within any number of days that are suitable for the compositions as described herein.

The present disclosure provides compositions comprising VSTs that exhibit negligible alloreactivity. In some embodiments, the compositions comprising VSTs that exhibit less activation induced cell death of antigen-specific T cells harvested from a patient than corresponding antigen-specific T cells harvested from the same patient. In some embodiments, the compositions are not cultured in the presence of both IL-7 and IL-4. In some embodiments, the compositions comprising VSTs exhibit viability of greater than 70%.

In some embodiments, the compositions are negative for bacteria and fungi for at least 1 days, at least 2 days, at least 3 days, at least 4 days, at least 5 days, at least 6 days at least 7 days, at least 8 days, at least 9 days, at least 10 days, in culture. In some embodiments, the composition is negative for bacteria and fungi for at least 7 days in culture. In some embodiments, the compositions exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In some embodiments, the compositions exhibit less than 5 EU/ml of endotoxin. In some embodiments, the compositions are negative for mycoplasma.

In some embodiments, the pepmixes used for constructing the polyclonal of VSTs are chemically synthesized. In some embodiments, the pepmixes are optionally >10%, >20%, >30%, >40%, >50%, >60%, >70%, >80%, or >90%, inclusive of all ranges and subranges therebetween, pure. In some embodiments, the pepmixes are optionally >90% pure.

In some embodiments, the VSTs are Th1 polarized. In some embodiments, the VSTs are able to lyse viral antigen-expressing targets cells. In some embodiments, the VSTs are able to lyse other suitable types of antigen-expressing targets cells. In some embodiments, the VSTs in the compositions do not significantly lyse non-infected autologous target cells. In some embodiments, the VSTs in the compositions do not significantly lyse non-infected autologous allogenic target cells.

The present disclosure provides pharmaceutical compositions comprising any compositions formulated for intravenous delivery. In some embodiments, the compositions are negative for bacteria for at least 1, at least 2, at least 3, at least 4, at least 5, at least 6, at least 7, at least 8 days, at least 9 days, at least 10 days, in culture. In some embodiments, the compositions are negative for bacteria for at least 7 days in culture. In some embodiments, the compositions are negative for fungi for at least 1, at least 2, at least 3, at least 4, at least 5, at least 6, at least 7, at least 8 days, at least 9 days, at least 10 days, in culture. In some embodiments, the compositions are negative for fungi for at least 7 days in culture.

In some embodiments, the present pharmaceutical compositions exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In some embodiments, the present pharmaceutical compositions are negative for mycoplasma.

The present disclosure also provides methods of treating or preventing viral infections comprising administering to a subject one or more effective dose of a VST disclosed herein (such as, e.g., a multi-R-VST disclosed herein that has specificity for PIV, influenza, RSV, and hMPV). The present disclosure also provides compositions (e.g., pharmaceutical compositions) comprising any of the VSTs disclosed herein (such as, e.g., a multi-R-VST disclosed herein that has specificity for PIV, influenza, RSV, and hMPV) and methods treating or preventing viral infections comprising administering to a subject one or more effective doses of such a pharmaceutical composition comprising a VST disclosed herein.

### Generation of Pepmix Libraries

In some embodiments of the disclosure, a library of peptides is provided to PBMCs ultimately to generate VSTs. The library in particular cases comprises a mixture of peptides (“pepmixes”) that span part or all of the same antigen. Pepmixes utilized in the disclosure may be from commercially available peptide libraries comprising peptides that are 15 amino acids long and overlapping one another by 11 amino acids, in certain aspects. In some cases, they may be generated synthetically. Examples include those from JPT Technologies (Springfield, Va.) or Miltenyi Biotec (Auburn, Calif.). In particular embodiments, the peptides are at least 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and in specific embodiments there is overlap of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length, for example.

In some embodiments, the amino acids as used in the pepmixes have at least 85%, at least 86%, at least 87%, at least 88%, at least 89%, at least 90%, at least 91%, at least 92%, at least 93%, at least 94%, at least 95%, at least 96%, at least 97%, at least 98%, at least 99, at least 99.9% purity, inclusive of all ranges and subranges therebetween. In some embodiments, the amino acids as used here in the pepmixes have at least 70% purity.

The mixture of different peptides may include any ratio of the different peptides, although in some embodiments each particular peptide is present at substantially the same numbers in the mixture as another particular peptide. The methods of preparing and producing pepmixes for multiviral cytotoxic T cells with broad specificity is described in US2018/0187152, which is incorporated by reference in its entirety.

### Production of VSTs

In some embodiments, methods of producing VSTs comprise isolating mononuclear cells (MNCs), or having MNCs, isolated, from blood obtained from donors. In some embodiments, the MNCs are PBMCs. MNCs and PBMCs are isolated by using the methods known by a skilled person in the art. By way of example, density centrifugation (gradient) (Ficoll-Paque) can be used for isolating PBMCs. In other example, cell preparation tubes (CPTs) and SepMate tubes with freshly collected blood can be used for isolating PBMCs.

In some embodiments, the MNCs are PBMCs. By way of example, PBMC can comprise lymphocytes, monocytes, and dendritic cells. By way of example, lymphocytes can include T cells, B cells, and NK cells. In some embodiments, the MNCs as used herein are cultured or cryopreserved. In some embodiments, the process of culturing or cryopreserving the cells can include contacting the cells in culture with one (or a portion of one) or more antigens under suitable culture conditions to stimulate and expand antigen-specific T cells. In some embodiments, the one or more antigen can comprise one or more viral antigen.

In some embodiments, the process of culturing or cryopreserving the cells can include contacting the cells in culture with one or more epitopes from one or more antigens under suitable culture conditions. In some embodiments, contacting the MNCs or PBMCs with one or more antigens, or one or more epitopes from one or more antigens, stimulate and expand a polyclonal population of antigen-specific T cells from each of the respective donor's MNCs or PMBCs. In some embodiments, the antigen-specific T cell lines can be cryopreserved.

In some embodiments, the one or more antigens can be in the form of a whole protein. In some embodiments, the one or more antigen can be a pepmix comprising a series of overlapping peptides spanning part of or the entire sequence of each antigen. In some embodiments, the one or more antigens can be a combination of a whole protein and a pepmix comprising a series of overlapping peptides spanning part of or the entire sequence of each antigen.

In some embodiments, the culturing of the PBMCs or MNCs is in a vessel comprising a gas permeable culture surface. In one embodiment, the vessel is an infusion bag with a gas permeable portion or a rigid vessel. In one embodiment, the vessel is a G-Rex® bioreactor. In one embodiment, the vessel can be any container, bioreactor, or the like, that are suitable for culturing the PBMCs or MNCs as described herein.

In some embodiments, the PBMCs or MNCs are cultured in the presence of one or more cytokines. In some embodiments, the cytokine is IL4. In some embodiments, the cytokine is IL7. In some embodiments, the cytokine is IL4 and IL7. In some embodiments, the cytokine includes IL4 and IL7, but not IL2. In some embodiments, the cytokine can be any combinations of cytokines that are suitable for culturing the PBMCs or MNCs as described herein.

In some embodiments, culturing the MNCs or PBMCs can be in the presence of at least 2, 3, 4, 5, 6, 7, 8, 9, 10, 11, 12, 13, 14, 15, 16, 17, 18, 19, 20, or more different pepmixes. Pepmixes, a plurality of peptides, comprise a series of overlapping peptides spanning part of or the entire sequence of an antigen. In some embodiments, the MNCs or PBMCs can be cultured in the presence of a plurality of pepmixes. In this instance, each pepmix covers at least one antigen that is different than the antigen covered by each of the other pepmixes in the plurality of pepmixes. In some embodiments, at least 2, 3, 4, 5, 6, 7, 8, 9, 10, 11, 12, 13, 14, 15, 16, 17, 18, 19, 20 or more different antigens are covered by the plurality of pepmixes. In some embodiments, at least one antigen from at least 2 different viruses are covered by the plurality of pepmixes.

In some embodiments, the pepmix comprises 15 mer peptides. In some embodiments, the pepmix comprises peptides that are suitable for the methods as described herein. In some embodiments, the peptides in the pepmix that span the antigen overlap in sequence by 8 amino acids, 9 amino acids, 10 amino acids, 11 amino acids, 12 amino acids, 13 amino acids, 14 amino acids, 15 amino acids. In some embodiments, the peptides in the pepmix that span the antigen overlap in sequence by 11 amino acids.

In some embodiments, the PBMCs or MNCs are cultured in the presence of pepmixes spanning influenza A antigen NP1 and Influenza A antigen MP1, RSV antigens N and F, hMPV antigens F, N, M2-1, and M, and PIV antigens M, HN, N, and F. In some embodiments, the PBMCs or MNCs are cultured in the presence of pepmixes spanning influenza A antigen NP1 and Influenza A antigen MP1, RSV antigens N and F, hMPV antigens F, N, M2-1, and M, and PIV antigens M, HN, N, and F and one or more coronavirus (e.g., SARS-CoV or SARS-CoV2) antigen disclosed herein. In some embodiments, the PBMCs or MNCs are cultured in the presence of pepmixes spanning EBV antigens LMP2, EBNA1, and BZLF1, CMV antigens IE1 and pp65, adenovirus antigens Hexon and Penton, BK virus antigens VP1 and large T, and HHV6 antigens U90, U11, and U14. In some embodiments, the antigen specific T cells are tested for antigen-specific cytotoxicity.

The present disclosure provides methods of lysing a target cell comprising contacting the target cell with the compositions or pharmaceutical compositions as described herein. In some embodiments, the contacting between the target cell and the compositions or pharmaceutical compositions occurs in vivo in a subject. In some embodiments, the contacting between the target cell and the compositions or pharmaceutical compositions occurs in vivo via administration of the VSTs to a subject. In some embodiments, the subject is a human.

The present disclosure provides methods of treating or preventing a viral infection comprising administering to a subject in need thereof the compositions or the pharmaceutical compositions as described herein. In some embodiments, the VSTs are administered to a subject at between 5×10³ and 5×10⁹ VSTs/m², 5×10⁴ and 5×10⁸ VSTs/m², 5×10⁵ and 5×10⁷ VSTs/m², 5×10⁴ and 5×10⁸ VSTs/m², 5×10⁶ and 5×10⁹ VSTs/m², inclusive of all ranges and subranges therebetween. In some embodiments, the VSTs are administered to the subject. In some embodiments, the subject is immunocompromised. In some embodiments, a subject that has a PIV infection is administered the multi-R-VSTs disclosed herein that are specific for PIV, RSV, hMPV, and influenza. In some embodiments, the multi-R-VSTs have cross-over specificity such that they are efficacious against viral infections that differ from the virus from which they were generated. For example, but not to be limited by example, in some embodiments, the PIV specific VSTs in the multi-R-VSTs are generated against PIV3 antigens. In some embodiments, the PIV infection that is treated is PIV3. In some embodiments the PIV infection that is treated is a serotype other than PIV3. In some embodiments, a subject that has a RSV infection is administered the multi-R-VSTs disclosed herein that are specific for PIV, RSV, hMPV, and influenza. In some embodiments, a subject that has a hMPV infection is administered the multi-R-VSTs disclosed herein that are specific for PIV, RSV, hMPV, and influenza. In some embodiments, a subject that has an influenza infection is administered the multi-R-VSTs disclosed herein that are specific for PIV, RSV, hMPV, and influenza. In some embodiments, a subject that has a coronavirus (e.g., SARS-CoV or SARS-CoV2) infection is administered multi-R-VSTs disclosed herein that are specific for PIV, RSV, hMPV, influenza, and a coronavirus (e.g., SARS-CoV or SARS-CoV2).

In some embodiments, the subject can have one or more medical conditions. In some embodiments, the subject receives a matched related donor transplant with reduced intensity conditioning prior to receiving the VSTs. In some embodiments, the subject receives a matched unrelated donor transplant with myeloablative conditioning prior to receiving the VSTs. In some embodiments, the subject receives a haplo-identical transplant with reduced intensity conditioning prior to receiving the VSTs. In some embodiments, the subject receives a matched related donor transplant with myeloablative conditioning prior to receiving the VSTs. In some embodiments, the subject has received a solid organ transplantation. In some embodiments, the subject has received chemotherapy. In some embodiments, the subject has an HIV infection. In some embodiments, the subject has a genetic immunodeficiency. In some embodiments, the subject has received an allogeneic stem cell transplant. In some embodiments, the subject has a preexisting condition that renders them more susceptible to getting a viral infection and/or to having a significant adverse outcome following a viral infection. For example, in some embodiments, the subject has cardiovascular disease. In some embodiments, the subject has diabetes. In some embodiments, the subject has chronic respiratory disease. In some embodiments, the subject has hypertension. In some embodiments, the subject has cancer. In some embodiments, the subject is obese. In some embodiments, the subject is elderly. In some embodiments, the subject has more than one medical conditions as described in this paragraph. In some embodiments, the subject has all medical conditions as described in this paragraph. In some embodiments, the patient is infected with a coronavirus (e.g., SARS-CoV or SARS-CoV2). In some embodiments, the patient has been diagnosed with COVID-19. In some embodiments, the patient is immunocompromised. As used herein, immunocompromised means having a weakened immune system. For example, patients who are immunocompromised have a reduced ability to fight infections and other diseases. In some embodiments, the patient is immunocompromised due to a treatment the patient received to treat the disease or condition or another disease or condition. In some embodiments, the cause of immunocompromised is due to age. In one embodiment, the cause of immunocompromised is due to young age. In one embodiment, the cause of immunocompromised is due to old age. In some embodiments, the patient is in need of a transplant therapy. In some embodiments, the subject has no other medical conditions other than infection with a coronavirus (e.g., SARS-CoV or SARS-CoV2). In some embodiments, the subject has acute myeloid leukemia, acute lymphoblastic leukemia, or chronic granulomatous disease.

In some embodiments, the treatment efficacy is measured post-administration of the VST cell line. In other embodiments, the treatment efficacy is measured based on viremic resolution of infection. In other embodiments, the treatment efficacy is measured based on viruric resolution of infection. In other embodiments, the treatment efficacy is measured based on resolution of viral load in a sample from the patient. In other embodiments, the treatment efficacy is measured via chest imaging to follow resolution of the disease in the lungs. In some embodiments, the sample is from a nasal swab. In other embodiments, the treatment efficacy is measured based on viremic resolution of infection, viruric resolution of infection, and resolution of viral load in a sample from the patient. In some embodiments, the treatment efficacy is measured by monitoring viral load detectable in the peripheral blood of the patient. In some embodiments, the treatment efficacy comprises resolution of macroscopic hematuria. In some embodiments, the treatment efficacy comprises reduction of hemorrhagic cystitis symptoms as measured by the CTCAE-PRO or similar assessment tool that examines patient and/or clinician-reported outcomes.

In some embodiments, a sample is selected from a tissue sample from the patient. In some embodiments, the sample is selected from a fluid sample from the patient. In some embodiments, the sample is selected from cerebral spinal fluid (CSF) from the patient. In some embodiments, the sample is selected from BAL from the patient. In some embodiments, the sample is selected from stool from the patient.

In some embodiments, the composition as described herein is administered to the subject a plurality of times. In some embodiments, the composition as described herein is administered to the subject more than one time. In some embodiments, the composition as described herein is administered to the subject more than two times. In some embodiments, the composition as described herein is administered to the subject more than three times. In some embodiments, the composition as described herein is administered to the subject more than four times. In some embodiments, the composition as described herein is administered to the subject more than five times. In some embodiments, the composition as described herein is administered to the subject more than six times. In some embodiments, the composition as described herein is administered to the subject more than seven times. In some embodiments, the composition as described herein is administered to the subject more than eight times. In some embodiments, the composition as described herein is administered to the subject more than nine times. In some embodiments, the composition as described herein is administered to the subject more than ten times. In some embodiments, the composition as described herein is administered to the subject a number of times that are suitable for the subjects. When multiple administrations of a composition are provided to an individual, the duration between administrations may be of any suitable length, including 1-24 hours, 1-7 days, 1-4 weeks, 1-12 months, or longer, and inclusive of all ranges and subranges therebetween.

In some embodiments, two or more compositions described herein comprising polyclonal populations of VSTs (e.g., multi-R-VSTs) are administered to the subject in combination. The two or more compositions may be administered to the subject sequentially or simultaneously. The two or more compositions may be pooled and administered as a single composition. The two or more compositions may be administered at separate times as separate compositions. In one embodiment, a subject is administered a first multi-R-VST composition comprising a polyclonal population of VSTs with specificity for PIV, influenza, RSV, and hMPV and the subject is also administered a second separate VST composition comprising a polyclonal population of VSTs with specificity for another virus. In particular embodiments, the other virus is a coronavirus (e.g., SARS-CoV2). In some embodiments a subject is administered a single composition comprising a pool of a first multi-R-VST composition comprising a polyclonal population of VSTs with specificity for PIV, influenza, RSV, and hMPV and a second VST composition comprising a polyclonal population of VSTs with specificity for another virus. In particular embodiments, the other virus is a coronavirus (e.g., SARS-CoV2). In some embodiments, the other virus is selected from BV, CMV, AdV, BK, JC virus, HHV6, RSV, Influenza, Parainfluenza, Bocavirus, Coronavirus, Rhinovirus, LCMV, Mumps, Measles, hMPV, Parvovirus B, Rotavirus, Merkel cell virus, herpes simplex virus, HPV, HIV, HTLV1, HHV8, Hepatitis C, Hepatitis B, HTLV1, Herpes simplex virus, West Nile Virus, zika virus, and Ebola.

In some embodiments, the administration of the composition effectively treats or prevents a viral infection in the subject. In some embodiments, the viral infection is PIV. In some embodiments, the viral infection is PIV3. In some embodiments, the viral infection is RSV. In some embodiments, the viral infection is Influenza. In some embodiments, the viral infection is hMPV. In some embodiments the viral infection is a coronavirus (e.g., SARS-CoV or SARS-CoV2). In some embodiments the viral infection is SARS-CoV. In some embodiments the viral infection is MERS-CoV. In some embodiments the viral infection is HCoV-HKU1. In some embodiments the viral infection is, and HCoV-0C43. In some embodiments the viral infection is HCoV-E229. In some embodiments the viral infection is HCoV-NL63.

The present disclosure provides compositions comprising a polyclonal population of VSTs that recognize a plurality of viral antigens. The present disclosure provides that the plurality of viral antigens comprise at least one antigen. In some embodiments, the at least one antigen can be a coronavirus (e.g., SARS-CoV or SARS-CoV2). In some embodiments, the at least one antigen can be from PIV. In some embodiments, the at least one antigen can be an RSV antigen. In some embodiments, the at least one antigen can be from Influenza. In some embodiments, the at least one antigen can be from hMPV.

In some embodiments, the present disclosure provides a polyclonal population of VSTs that recognize a plurality of viral antigens comprising at least one antigen from each of PIV, RSV, Influenza, and hMPV. In some embodiments, the present disclosure provides a polyclonal population of VSTs that recognize a plurality of viral antigens comprising the plurality of viral antigens comprise at least two antigens from each of PIV, RSV, Influenza, and hMPV. In some embodiments, the plurality of antigens comprise PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens can be selected from any of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. In some embodiments, the polyclonal population of VSTs is administered to a patient infected with influenza, RSV, PIV, and/or hMPV.

In at least some methods of the disclosure, the VSTs generated are administered to an individual, for example, an immunocompromised individual. In some cases, the individual has had or will be having allogeneic stem cell transplant. In specific embodiments, the cells are administered by injection, such as intravenous, intramuscular, intradermal, subcutaneous, intraperitoneal injection, and so forth, for example. In some embodiments, the individual has lymphoma or leukemia. In some embodiments, the VSTs are further defined as polyclonal CD4+ and CD8+VSTs. The PBMCs may be allogeneic to the individual or autologous to the individual. In some embodiments, the methods of the invention further comprise the step of exposing the VSTs to one or more compositions that stimulate cell division, such as phytohemagglutinin; in some aspects the compound is a mitogen.

In some embodiments, the present disclosure provides pharmaceutical compositions comprising the compositions as described herein formulated for intravenous delivery. In some embodiments, the present disclosure provides pharmaceutical compositions comprising a population of VSTs disclosed herein and one or more carriers, excipients, diluents, buffers, and/or delivery vehicles. In some particular embodiments, the present disclosure provides pharmaceutical compositions comprising one or more VST composition described herein formulated for intravenous delivery. In certain embodiments, the compositions that are formulated for intravenous delivery may comprise one or more of the expanded VSTs disclosed herein suspended or resuspended in their culture media. The compositions that are formulated for intravenous delivery may additionally or alternatively comprise one or the expanded VSTs resuspended in a suitable carrier, excipient, diluent, buffer, and/or delivery vehicle. In certain embodiments, the compositions that are formulated for intravenous delivery may comprise one or more of the expanded VSTs disclosed herein resuspended in saline. In some embodiments, the composition as described herein is negative for bacteria. In some embodiments, the composition as described herein is negative for fungi. In some embodiments, the composition as described herein is negative for bacteria or fungi for at least 1 days, at least 2 days, at least 3 days, at least 4 days, at least 5 days, at least 6 days, at least 7 days, at least 8 days, at least 9 days, at least 10 days, in culture. In some embodiments, the composition as described herein is negative for bacteria or fungi for at least 7 days in culture.

In some embodiments, the pharmaceutical compositions formulated for intravenous delivery exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In some embodiments, the pharmaceutical compositions formulated for intravenous delivery are negative for mycoplasma.

### EXAMPLES

### Example 1

### Methods

Unless otherwise indicated, the Examples provided below utilized the following materials and methods.

### Flow Cytometry

### Immunophenotyping

Multi-R-VSTs were surface-stained with monoclonal antibodies to: CD3, CD25, CD28, CD45RO, CD279 (PD-1) [Becton Dickinson (BD), Franklin Lakes, N.J.], CD4, CD8, CD16, CD62L, CD69 (Beckman Coulter, Brea, Calif.) and CD366 (TIM-3) (BioLegend, San Diego, Calif.). Cells were pelleted in phosphate-buffered saline (PBS) (Sigma-Aldrich), then antibodies added in saturating amounts (5 μl) followed by incubation for 15 mins at 4° C. Subsequently, cells were washed, resuspended in 300 μl of PBS and at least 20,000 live cells acquired on a Gallios™ Flow Cytometer and analyzed with Kaluza® Flow Analysis Software (Beckman Coulter).

### Intracellular Cytokine Staining (ICS)

Multi-R-VSTs were harvested, resuspended in VST medium (2×106/ml) and 200 μl added per well of a 96-well plate. Cells were incubated overnight with 200 ng of individual test or control (irrelevant non-viral, e.g. SURVIVIN, WT1) pepmixes along with Brefeldin A (1 μg/ml), monensin (1 μg/ml), CD28 and CD49d (1 μg/ml) (BD). Next, VSTs were washed with PBS, pelleted, surface-stained with CD8 and CD3 (5 μl/antibody/tube) for 15 mins at 4° C., then washed, pelleted, fixed and permeabilized with Cytofix/Cytoperm solution (BD) for 20 mins at 4° C. in the dark. After washing with Perm/Wash Buffer (BD), cells were incubated with 10 μl of IFNγ and TNFα antibodies (BD) for 30 min at 4° C. in the dark. Cells were then washed twice with Perm/Wash Buffer and at least 50,000 live cells were acquired on a Gallios™ Flow Cytometer and analyzed with Kaluza® Flow Analysis Software.

### FoxP3 Staining

FoxP3 staining was performed using the eBioscience FoxP3 kit (Thermo Fisher Scientific, Waltham, Mass.), per manufacturers' instructions. Briefly, 1×106 cells were surface-stained with CD3, CD4 and CD25 antibodies, then washed, resuspended in 1 ml fixation/permeabilization buffer and incubated for 1 hour at 4° C. in the dark. After washing with PBS, cells were resuspended in permeabilization buffer, incubated with 5 μl isotype or FoxP3 antibody (Clone PCH101) for 30 minutes at 4° C., then washed and acquired on a Gallios™ Flow Cytometer followed by analysis with Kaluza® Flow Analysis Software.

### Functional Studies

### Enzyme-Linked Immunospot (ELIspot)

ELIspot analysis was used to quantitate the frequency of IFNγ and Granzyme B-secreting cells. Briefly, PBMCs and multi-R-VSTs were resuspended at 5×106 and 2×106 cells/ml, respectively in VST medium and 100 μl of cells was added to each ELIspot well. Antigen-specific activity was measured after direct stimulation (500 ng/peptide/ml) with the individual stimulating [NP1, MP1 (Influenza); N, F (RSV); F, N, M2-1, M (hMPV); M, HN, N, F (PIV)], or control pepmixes (Survivin, WT1). Staphylococcal Enterotoxin B (SEB) (1 μg/ml) and PHA (1 μg/ml) were used as positive controls for PBMCs and VSTs, respectively. After 20 hours of incubation, plates were developed as previously described, dried overnight at room temperature and then sent to Zellnet Consulting (New York) for quantification. Spot-forming cells (SFC) and input cell numbers were plotted and the specificity threshold for VSTs was defined as ≥30 SFC/2×105 input cells.

### Multiplex

The multi-R-VST cytokine profile was evaluated using the MILLIPLEX High Sensitivity Human Cytokine Panel (Millipore, Billerica, Mass.). 2×105 VSTs were stimulated with pepmixes (NP1, MP1,

N, F, F, N, M2-1, M, M, HN, N, and F) (1 μg/ml) overnight. Subsequently, supernatant was collected, plated in duplicate wells, incubated overnight at 4° C. with antibody-immobilized beads, then washed and plated for 1 hour at room temperature with biotinylated detection antibodies. Finally, streptavidin-phycoerythrin was added for 30 minutes at room temperature. Samples were washed and analyzed on a Luminex 200 (XMAP Technology) using the xPONENT software.

### Chromium Release Assay

A standard 4-hour chromium (Cr51) release assay was used to measure the specific cytolytic activity of multi-R-VSTs with autologous antigen-loaded PHA blasts as targets (20 ng/pepmix/1×106 target cells). Effector:Target (E:T) ratios of 40:1, 20:1, 10:1, and 5:1 were used to analyze specific lysis. The percentage of specific lysis was calculated [(experimental release−spontaneous release)/(maximum release−spontaneous release)]×100. In order to measure the autoreactive and alloreactive potential of multi-R-VST lines, autologous and allogeneic PHA blasts alone were used as targets.

### Example 2

### Generation of Polyclonal Multi-R-VSTs from Healthy Donors

In the present study we explored the feasibility of targeting multiple clinically problematic respiratory viruses using ex vivo expanded T cells. Specifically, we produced VSTs with specificity against Influenza, RSV, hMPV, and PIV and demonstrated clinical efficacy in transplant recipients who successfully controlled active infections.

### BACKGROUND

CARV-associated acute upper and lower RTIs are a major public health problem with young children, the elderly and those with suppressed or compromised immune systems being most vulnerable(1-3). These infections are associated with symptoms including cough, dyspnea, and wheezing and dual/multiple co-existing infections are common, with frequencies that may exceed 40% among children less than 5 years and are associated with increased risk of morbidity and hospitalization(22-26). Among immunocompromised allogeneic HSCT recipients up to 40% experience CARV infections that can range from mild (associated symptoms including rhinorrhea, cough and fever) to severe (bronchiolitis and pneumonia) with associated mortality rates as high as 50% in those with LRTIs(5-9). The therapeutic options are limited. For hMPV and PIV there are currently no approved preventative vaccines nor therapeutic antiviral drugs, while the off-label use of the nucleoside analog RBV and the investigational use of DAS-181 (a recombinant sialidase fusion protein) have had limited clinical impact(10, 11, 27, 28). The preventative annual Influenza vaccine is not recommended for allogeneic HSCT recipients until at least 6 months post-transplant (and excluded in recipients of intensive chemotherapy or anti-B-cell antibodies), while neuraminidase inhibitors are not always effective for the treatment of active infections(12). For RSV, aerosolized RBV is FDA-approved for the treatment of severe bronchiolitis in infants and children, and it is also used off-label for the prevention of upper or lower RTIs and treatment of RSV pneumonia in HSCT recipients(13, 15, 16). However, its widespread use is limited by the cumbersome nebulization device and ventilation system required for drug delivery as well as the considerable associated cost. For example, in 2015 aerosolized RBV cost $29,953 per day, with 5 days representing a typical treatment course(14). Thus, the lack of approved treatments combined with the high cost of antiviral agents led us to explore the potential for using adoptively-transferred T cells to prevent and/or treat CARV infections in this patient population.

The pivotal role of functional T cell immunity in mediating viral control of CARVs has only recently garnered attention. For example, a retrospective study of 181 HSCT patients with RSV URTIs, reported lymphopenia (defined as ALC ≤100/mm3) as a key determinant in identifying patients whose infections would progress to LRTI, while RSV neutralizing antibody levels were not significantly associated with disease progression(29). Furthermore, in a recent retrospective analysis of 154 adult patients with hematologic malignancies with or without HSCT treated for RSV LRTI, lymphopenia was significantly associated with higher mortality rates(30). Both of these studies are suggestive of the importance of cellular immunity in mediating protective immunity in vivo.

### Donors and Cell Lines

Peripheral blood mononuclear cells (PBMCs) were obtained from healthy volunteers and HSCT recipients with informed consent using Baylor College of Medicine IRB-approved protocols (H-7634, H-7666) and were used to generate phytohemagglutinin (PHA) blasts and multi-R-VSTs. PHA blasts were generated as previously reported(20) and cultured in VST medium [45% RPMI 1640 (HyClone Laboratories, Logan, Utah), 45% Click's medium (Irvine Scientific, Santa Ana, Calif.), 2 mM GlutaMAX TM-I (Life Technologies, Grand Island, N.Y.), and 10% human AB serum (Valley Biomedical, Winchester, Va.)] supplemented with interleukin 2 (IL2) (100 U/mL; NIH, Bethesda, Md.), which was replenished every 2 days.VST Generation

### Generation and Phenotypic Characterization of Multi-Respiratory Virus Specific T Cells

We generated virus specific T cell (VST) T cell lines containing sub-populations of cells reactive against Influenza, RSV, hMPV, and PIV by the following method:

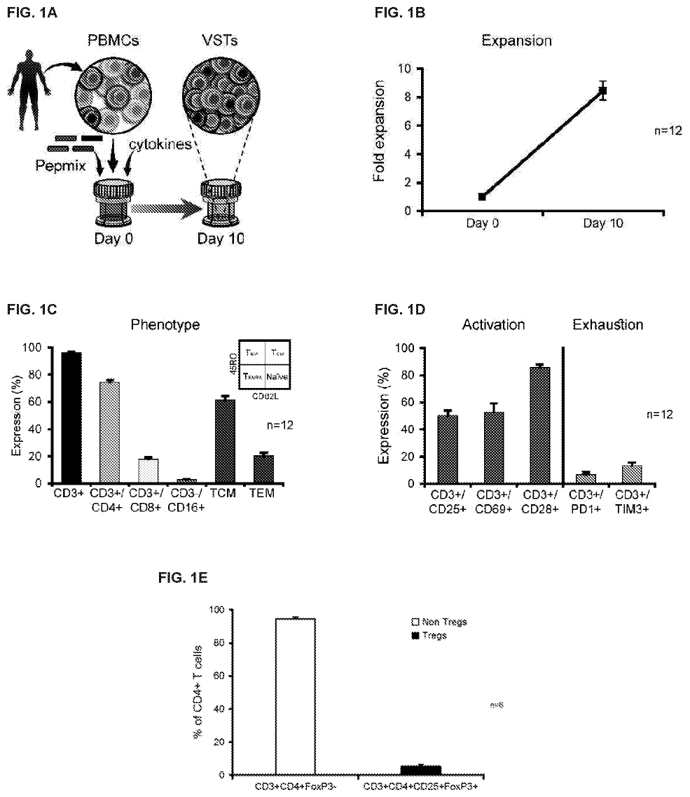

PBMCs (2.5×10⁷) were harvested as above and then transferred to a G-Rex10 (Wilson Wolf Manufacturing Corporation, St. Paul, Minn.) with 100 ml of VST medium supplemented with IL7 (20 ng/ml), IL4 (800 U/ml) (R&D Systems, Minneapolis, Minn.) and pepmixes (2 ng/peptide/ml) and cultured for 10-13 days at 37° C., 5% CO2 (FIG. 1A).

The pepmixes were peptide libraries (15 mers overlapping by 11aa) spanning Influenza A antigens (NP1, MP1), RSV antigens (N, F), hMPV antigens (F, N, M2-1, M) (JPT Peptide Technologies, Berlin, Germany) and antigens PIV antigens (M, HN, N, F) (Genemed Synthesis, San Antonio, Tex.). Lyophilized pepmixes were reconstituted in Dimethyl sulfoxide (DMSO) (Sigma-Aldrich) and stored at −80° C. until use.

### Results

Over 10-13 days we achieved an average 8.5 fold increase in cells (FIG. 1B) [increase from 0.25×10⁷ PBMCs/cm² to mean 1.9±0.2×10⁷ cells/cm² (median: 2.05×10⁷, range: 0.6-2.82×10⁷ cells/cm²; n=12). We used flow cytometry to immunophenotype the expanded cells as described above. The expanded cells were comprised almost exclusively of CD3+ T cells (96.2±0.6%; mean±SEM), with a mixture of cytotoxic (CD8+; 18.1±1.3%) and helper (CD4+; 74.4±1.7%) T cells [FIG. 1C] with no evidence of regulatory T cell outgrowth, as assessed by CD4/CD25/FoxP3+ staining [FIG. 1E]. Furthermore, the expanded cells displayed a phenotype consistent with effector function and long term memory as evidenced by upregulation of the activation markers CD25 (50.2±3.8%), CD69 (52.8±6.3%), CD28 (85.8±2%) as well as expression of central (CD45RO+/CD62L+: 61.4±3%) and effector memory markers (CD45RO+/CD62L−: 20.3±2.3%), with minimal PD1 (6.9±1.4%) or Tim3 (13.5±2.3%) surface expression [FIGS. 1C-1D].

Thus, the methods disclosed herein result in the rapid expansion of a polyclonal population of activated cytotoxic and helper T cells with no signs of exhaustion suggesting the expansion of VSTs with specificity for the respiratory virus antigens.

### Example 3

### Characterization of Anti-Viral Specificity of Multi-R-VSTs

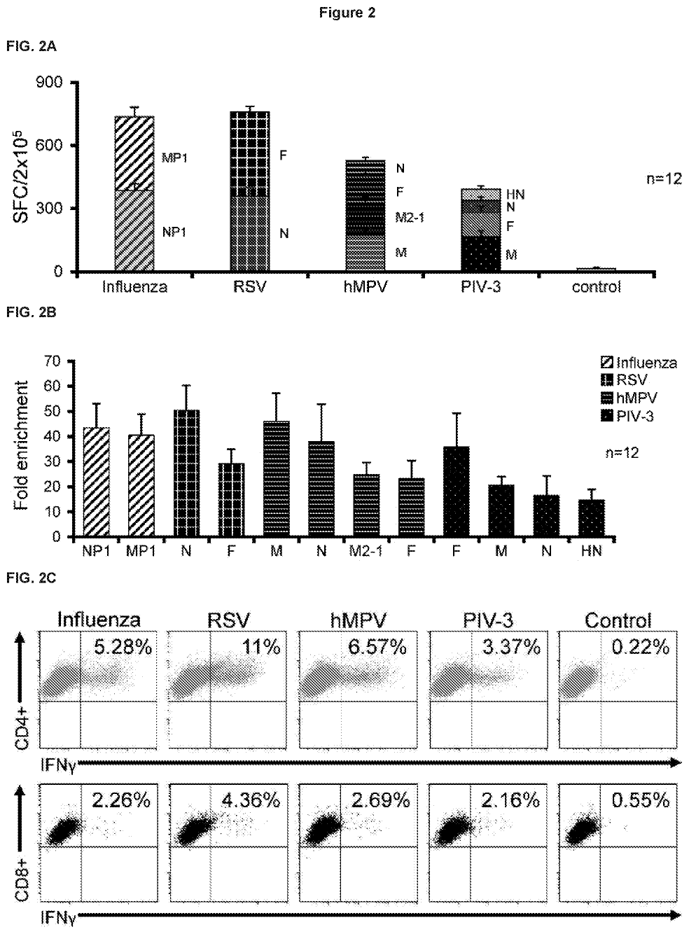

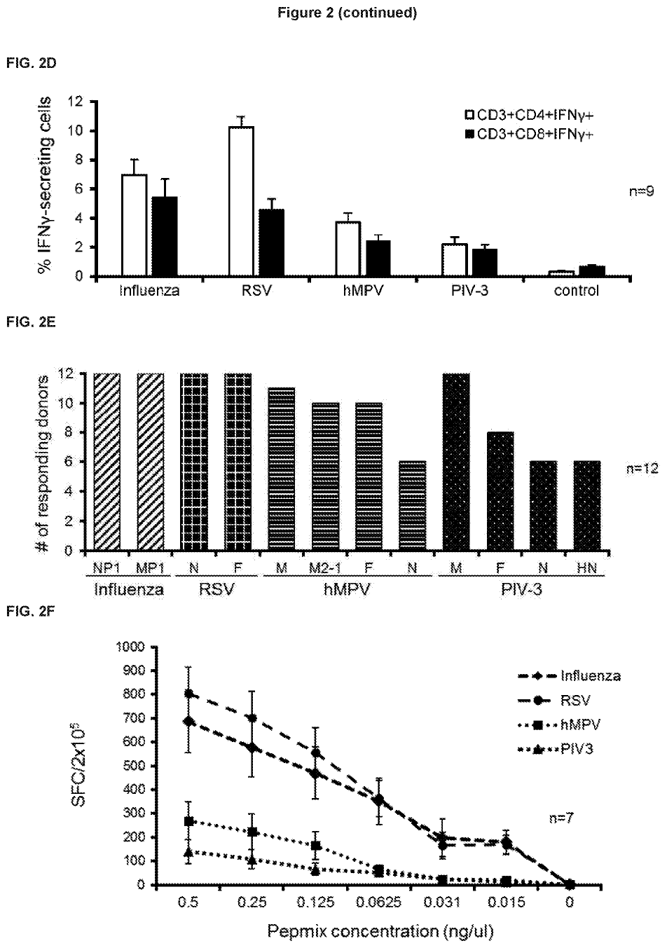

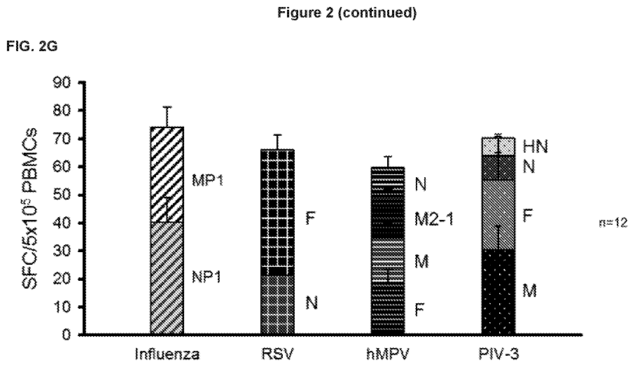

To next determine whether the expanded populations were antigen-specific we performed an IFNγ and Granzyme B-secreting cells ELIspot assay, using each of the individual stimulating antigens as an immunogen. The ELIspot analysis was performed as discussed above. All 12 lines generated proved to be reactive against all of the target viruses [Table 1, FIG. 2E].

TABLE 1

| | Reactivity of expanded VST lines against individual stimulating antigens. | | | | | | | | | | | |
|---|---|---|---|---|---|---|---|---|---|---|---|---|
| | Influenza | | RSV | | hMPV | | | | PIV | | | |
| Donor | NP1 | MP1 | N | F | M | M2-1 | F | N | M | F | N | HN |
| 1 | ✓ | ✓ | ✓ | ✓ | ✓ | ✓ | ✓ | x | ✓ | x | ✓ | ✓ |
| 2 | ✓ | ✓ | ✓ | ✓ | ✓ | ✓ | ✓ | ✓ | ✓ | ✓ | ✓ | ✓ |
| 3 | ✓ | ✓ | ✓ | ✓ | ✓ | ✓ | ✓ | ✓ | ✓ | ✓ | ✓ | ✓ |
| 4 | ✓ | ✓ | ✓ | ✓ | ✓ | x | ✓ | ✓ | ✓ | ✓ | x | x |
| 5 | ✓ | ✓ | ✓ | ✓ | ✓ | ✓ | ✓ | ✓ | ✓ | ✓ | ✓ | ✓ |
| 6 | ✓ | ✓ | ✓ | ✓ | ✓ | ✓ | ✓ | x | ✓ | x | x | ✓ |
| 7 | ✓ | ✓ | ✓ | ✓ | ✓ | x | ✓ | x | ✓ | ✓ | x | x |
| 8 | ✓ | ✓ | ✓ | ✓ | ✓ | ✓ | ✓ | x | ✓ | ✓ | ✓ | x |
| 9 | ✓ | ✓ | ✓ | ✓ | ✓ | ✓ | ✓ | ✓ | ✓ | x | x | x |
| 10 | ✓ | ✓ | ✓ | ✓ | x | ✓ | x | x | ✓ | x | x | x |
| 11 | ✓ | ✓ | ✓ | ✓ | ✓ | ✓ | ✓ | ✓ | ✓ | ✓ | x | ✓ |
| 12 | ✓ | ✓ | ✓ | ✓ | ✓ | ✓ | x | ✓ | ✓ | ✓ | ✓ | x |

FIG. 2A summarizes the magnitude of activity against each of the stimulating antigens, while FIG. 2F shows the response of our expanded VSTs to titrated concentrations of viral antigen. Of note, over the 10-13 days in culture we achieved an enrichment in virus-specific T cells of between 14.6±4.3 (PIV-HN) and 50.4±9.9 fold (RSV-N) [FIG. 2B; the precursor frequencies of CARV-reactive T cells within donor PBMCs are summarized in FIG. 2G]. Taken together these data suggest that respiratory virus-specific T cells reside in the memory pool and can be readily amplified ex vivo using GMP-compliant manufacturing methodologies.

To next evaluate whether viral specificity was contained with the CD4+ or CD8+ or both T cell subsets we performed ICS, gating on CD4+ and CD8+ IFNγ-producing cells. FIG. 2C shows representative results from 1 donor with activity against all 4 viruses detected in both T cell compartments [(CD4+: Influenza—5.28%; RSV—11%; hMPV—6.57%; PIV—3.37%), (CD8+:Influenza—2.26%; RSV—4.36%; hMPV—2.69%; PIV—2.16%)] while FIG. 2D shows summary results for 9 donors screened, confirming that our multi-R-VST are polyclonal and poly-specific.

Thus, these data confirm that these methods product multi-R-VSTs that are polyclonal and comprise both CD4+ and CD8+ T cells.

### Example 4

### Assessment of In Vitro Efficacy of Multi-R-VSTs

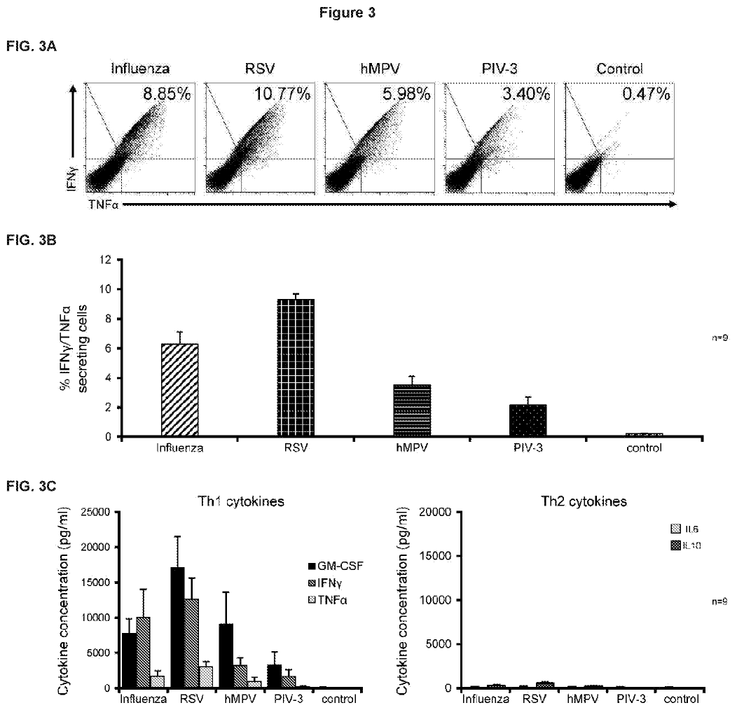

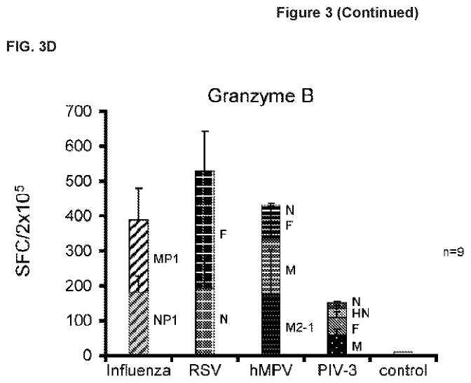

The production of multiple proinflammatory cytokines and expression of effector molecules has been shown to correlate with enhanced cytolytic function and improved in vivo T cell activity. Hence, we next examined the cytokine profile of our multi-R-VSTs following antigen exposure. As shown in FIG. 3, the majority of IFNγ-producing cells also produced TNFα [FIG. 3A—detailed ICS results from 1 donor; summary results for 9 donors; FIG. 3B], in addition to GM-CSF, as measured by Luminex array [FIG. 3C—left panel] with baseline levels of prototypic Th2/suppressive cytokines [FIG. 3C—right panel]. Furthermore, upon antigenic stimulation our cells produced the effector molecule Granzyme B, suggesting the cytolytic potential of these expanded cells [FIG. 3D, n=9]. Taken together, this data demonstrates the Th1-polarized and polyfunctional characteristics of our multi-R-VSTs.

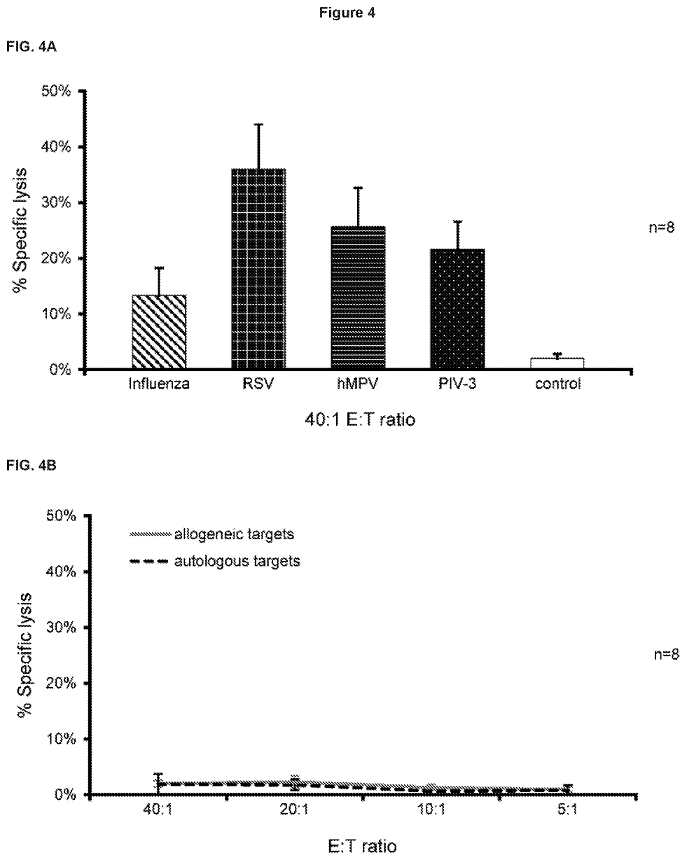

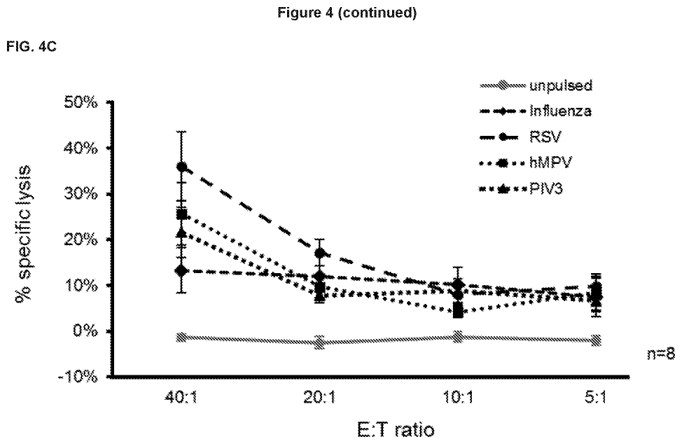

To investigate the cytolytic potential of these expanded cells in vitro we co-cultured multi-R-VSTs with autologous Cr⁵¹-labeled PHA blasts, which were loaded with viral pepmixes with unloaded PHA blasts serving as a control. As shown in FIG. 4A and FIG. 4C, viral antigen-loaded targets were specifically recognized and lysed by our expanded multi-R-VSTs (40:1 E:T—Influenza: 13±5%, RSV: 36±8%, hMPV: 26±7%, PIV: 22±5%, n=8). Finally, even though these VSTs had received only a single stimulation there was no evidence of activity against non-infected autologous targets nor of alloreactivity (graft versus host potential) using HLA-mismatched PHA blasts as targets [FIG. 4B]. This is an important consideration if these cells are to be administered to allogeneic HSCT recipients.

Thus, the multi-R-VSTs possess in vitro efficacy and are safe.

### Example 5

### Assessment of In Vivo Efficacy of Multi-R-VSTs

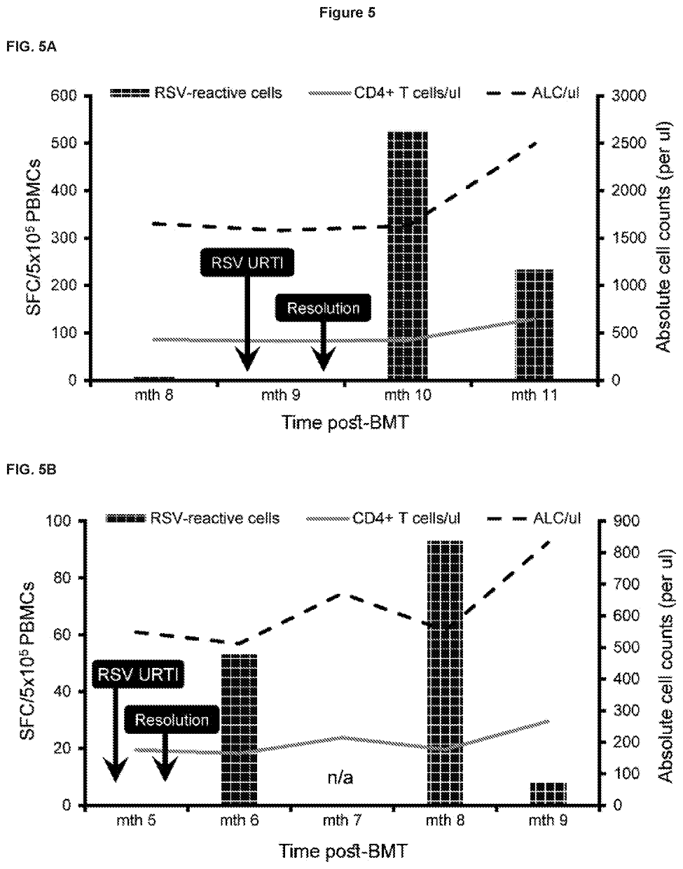

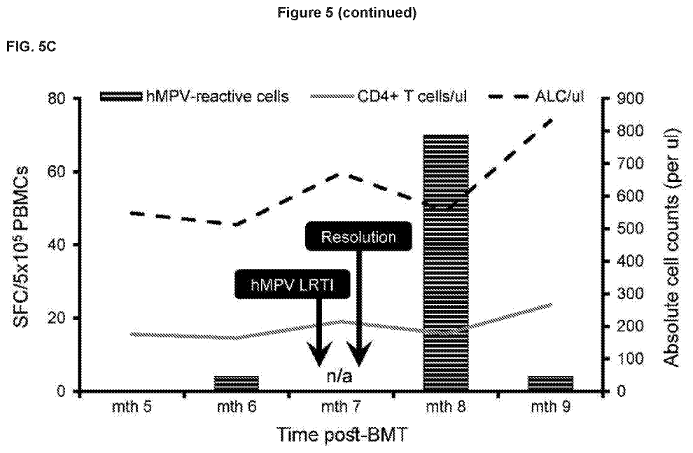

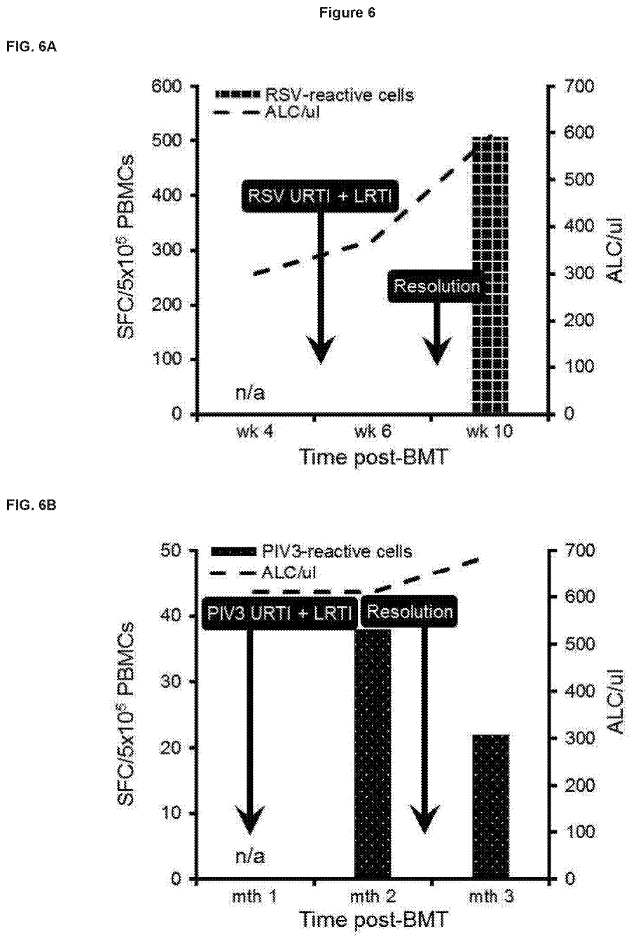

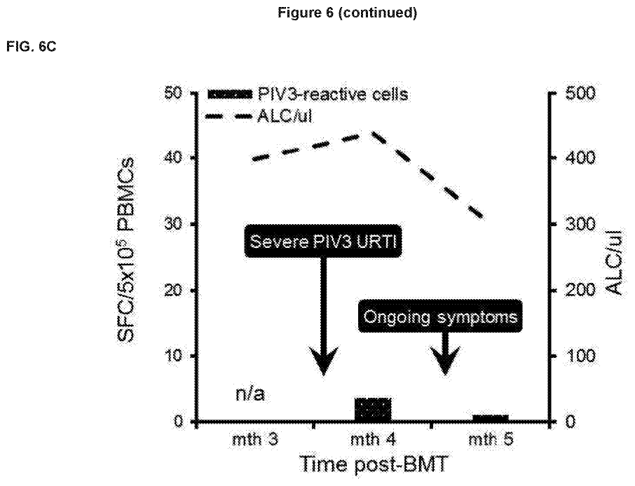

To assess the potential clinical relevance of multi-R-VSTs we investigated whether allogeneic HSCT recipients with active/recent CARV infections exhibited elevated levels of reactive T cells during/following an active viral episode. FIG. 5A shows the results of Patient #1, a 64-year old male with acute myeloid leukemia (AML) who received a matched related donor (MRD) transplant with reduced intensity conditioning. The patient developed a severe URTI 9 months post-HSCT that was confirmed to be RSV-related by PCR analysis. He was not on any immunosuppression at the time of infection but was placed on prednisone the day of infection diagnosis to control pulmonary inflammation. Within 4 weeks his symptoms resolved without specific antiviral treatment. To assess whether T cell immunity contributed to viral clearance, we analyzed the circulating frequency of RSV-specific T cells over the course of his infection. Immediately prior to infection this patient exhibited a very weak response to the RSV antigens N and F (6.5 SFC/5×10⁵ PBMCs). However, within a month of viral exposure, RSV-specific T cells had expanded in vivo (527 SFC/5×10⁵ PBMCs), representing an 81-fold increase in reactive cells, as seen in FIG. 5A, which declined thereafter, coincident with viral clearance. Of note, the observed RSV-specific responses did not follow the overall increase in lymphocyte/CD4+ counts, thus indicating that T cell expansion was virus-driven and not due to general immune reconstitution. Similarly, Patient #2, a 23-year old male with acute lymphoblastic leukemia (ALL) who received a matched unrelated donor (MUD) transplant with myeloablative conditioning, and developed a severe RSV-related URTI 5 months post HSCT while on tapering doses of tacrolimus. His infection symptomatically resolved within 1 week, coincident with the administration of ribavirin. To investigate whether endogenous immunity also played a role in viral clearance we monitored reactive T cell numbers over time. As seen in FIG. 5B, viral clearance was accompanied by an increase in the circulating frequency of RSV-specific T cells (peak 93 SFC/5×10⁵ PBMCs) with subsequent return to baseline levels. The same patient was hospitalized 7 months post-transplant for a subsequent pneumococcal pneumonia with concurrent detection (by PCR) of hMPV in sputum. His pneumonia was treated with antibiotics with subsequent resolution of disease and viral clearance, coincident with a marked expansion of hMPV-specific T cells (reactive against F, N, M2-1 and M), which increased from 4 SFC to a peak of 70 SFC and subsequent decline to baseline levels [FIG. 5C]. Again, the observed RSV- and hMPV-specific responses were independent of the overall increase in lymphocyte/CD4+ counts. FIG. 6 shows the results of 3 additional HSCT recipients who developed CARV infections. Patient #3, is a 15-year old female with AML who received a haplo-identical transplant with reduced intensity conditioning, and developed an RSV-induced URTI and LRTI while on tacrolimus 5 weeks post-transplant. The patient was administered ribavirin and the infection resolved within 4 weeks. We monitored RSV-reactive T cells over time and, as can be seen in FIG. 6A, viral clearance coincided with a striking increase in the frequency of RSV-specific T cells (from 0 to 506 SFC/5×10⁵ PBMCs). Similarly, Patient #4, a 10-year old male patient with ALL who received a MUD transplant with myeloablative conditioning, developed a PIV3-related URTI and LRTI 1 month after HSCT while on tacrolimus. His infection symptomatically resolved within 5 weeks, coincident with the administration of ribavirin. To investigate whether endogenous immunity also played a role in viral clearance, we monitored PIV3-reactive T cell numbers over time. As seen in FIG. 6B, viral clearance was accompanied by an increase in the circulating frequency of T cells specific for the PIV3 antigens M, HN, N and F (peak 38 SFC/5×10⁵ PBMCs) with subsequent decline. Finally, we show Patient #5, a 3-year old male with chronic granulomatous disease who received a MRD transplant with myeloablative conditioning and developed a severe PIV3-related URTI 4 months post-HSCT while on cyclosporine. The patient received ribavirin but (at last timepoint assessed) continued to exhibit disease symptoms and failed to demonstrate PIV3-specific T cells (FIG. 6C). Taken together, these data suggest the in vivo relevance of CARV-specific T cells in the control of viral infections in immunocompromised patients.

### CONCLUSION

Respiratory viral infections due to community-acquired respiratory viruses (CARVs) including respiratory syncytial virus (RSV), influenza, parainfluenza virus (PIV) and human metapneumovirus (hMPV) are detected in up to 40% of allogeneic hematopoietic stem cell transplant (allo-HSCT) recipients, in whom they may cause severe disease such as bronchiolitis and pneumonia that can be fatal. Given the lack of approved antiviral agents for these CARVs and data demonstrating that adoptively transferred ex vivo-expanded virus-specific T cells (VSTs) can be clinically beneficial for the treatment of both latent [Epstein-Barr virus (EBV), cytomegalovirus (CMV), BK virus (BKV), human herpesvirus 6 (HHV6)] and lytic [adenovirus (AdV)] viruses in recipients of allo-HSCT, it was considered to explore the potential for extending this approach to at least Influenza, RSV, hMPV and PIV3.

Thus, the inventors exposed PBMCs from healthy donors to a cocktail of pepmixes (overlapping peptide libraries) spanning immunogenic antigens from certain target viruses [Influenza—NP1 and MP 1; RSV—N and F; hMPV—F, N, M2-1 and M; PIV3—M, HN, N and F] followed by expansion in the presence of activating cytokines in a G-Rex. Over 10-13 days the inventors achieved an average 8.5 fold expansion (increase from 0.25×107 PBMCs/cm2 to mean 1.9±0.2×107 cells/cm2; n=12). Cultures comprised almost exclusively CD3+ T cells (96.2±0.6%; mean±SEM), a mixture of cytotoxic (CD8+) and helper (CD4+) T cells, with a phenotype consistent with immediate effector function and long term persistence, as evidenced by upregulation of the activation markers CD25, CD69, and CD28 and expression of central (CD45RO+/CD62L+) and effector memory markers (CD45RO+/CD62Lᵢ), with minimal PD1 or Tim3. Anti-viral specificity of multi-respiratory-VSTs was tested in an IFNγELISpot assay using each of the individual stimulating antigens as an immunogen. All 12 lines screened were reactive against each of the target viruses [Influenza: mean 735±75.6 SFC/2×105, RSV: 758±69.8, hMPV: 526±100.8, PIV3: 391±93.7]. The expanded VSTs were Th1-polarized effector cells, as evidenced by production of TNFα, GM-CSF and Granzyme B, with only baseline levels of Th2/suppressive cytokines.

The cells were tested in a standard Cr51 release assay and were able to lyse viral pepmix-loaded autologous PHA blasts (40:1 E:T—Influenza: 13±5%, RSV: 36±8%, hMPV: 26±7%, PIV: 22±5%, n=8) with no evidence of auto- or alloreactivity, attesting to both their selectivity and their safety for clinical use in HSCT recipients.

Finally, to assess the clinical significance of these findings we examined the peripheral blood of 5 allogeneic HSCT recipients with active RSV, hMPV and PIV3 infections. Four of these patients successfully controlled the viruses within 1-5 weeks, coincident with an amplification of endogenous reactive T cells and subsequent return to baseline levels upon viral clearance, while one patient failed to mount an immune response against the infecting virus and has equally failed to clear the infection to date. This data suggests that the adoptive transfer of ex vivo expanded cells should be clinically beneficial in patients whose own cellular immunity is lacking.

In conclusion, the inventors have shown that it is feasible to rapidly generate a single preparation of polyclonal (CD4+ and CD8+) multi-respiratory (multi-R)-VSTs with specificity for 12 immunodominant antigens derived from 4 target viruses: Influenza, RSV, hMPV and PIV3 using GMP-compliant manufacturing methodologies. The expanded cells are Th1-polarized, polyfunctional and selectively able to react to and kill, viral antigen-expressing target cells with no activity against non-infected autologous or allogeneic targets, attesting to both their selectivity for viral targets and their safety for clinical use. In various embodiments, such multi-respiratory virus-targeted cells (multi-R-VSTs) will provide broad spectrum benefit to immunocompromised individuals with uncontrolled CARV infections including in immunocompromised individuals.

### Example 6

### Generation of Polyclonal RSV-Specific VSTs from Healthy Donors

RSV is a particularly dangerous respiratory disease. It contributes to greater than 57,000 hospitalizations of young children (<5 yrs) annually in the U.S. and leads to 177,000 hospitalizations and 14,000 deaths among adults (>65 yrs) annually in the U.S. (Centers for Disease Control and Prevention). RSV often progresses to lower respiratory tract infections causing disease such as pneumonia, which can be fatal (Paulsen and Danziger-Isakov, Clin Chest Med 38 (2017)), and it is a major cause of disease in immunocompromised individuals including patients that have received allogeneic hematopoietic stem cell or solid organ transplants. Moreover, although Ribavirin is FDA-approved to treat children with severe pneumonia caused by RSV, this treatment is costly, difficult to administer, is associated with toxicity issues, and is not approved for other patients groups. Thus, there is a great need in the art for effective RSV treatments.

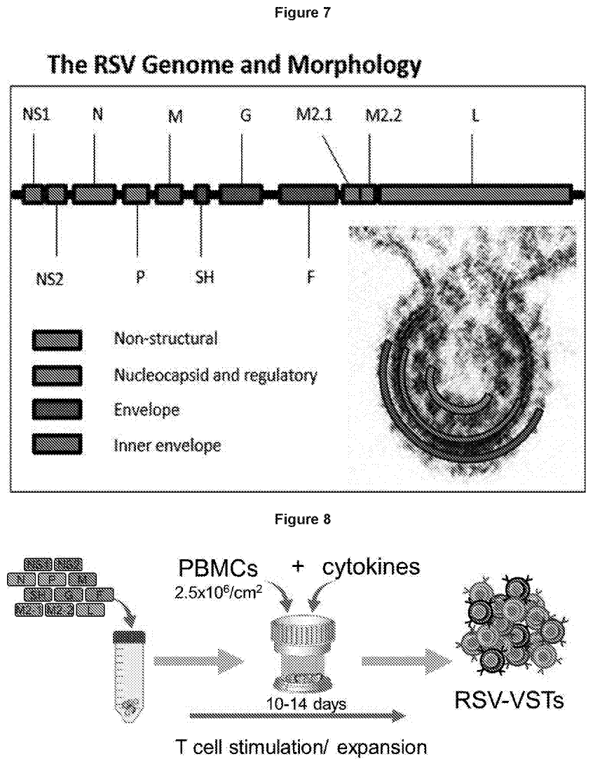

As shown in FIG. 7, in addition to RSV antigens N and F, which were included in the multi-R-VSTs described in the above Examples, the RSV genome also includes other antigens: G, M2-1, M, NS1, NS2, M2-2, P, L, and SH (FIG. 7). Having demonstrated the efficacy of our multi-R-VSTs for treating RSV infections, despite their only being generated with pepmixes RSV antigens N and F, we sought to investigate whether we could generate VSTs with specificity for a broader array of RSV antigens.

To that end, PBMCs were isolated as described in Example 1, and, as is shown in FIG. 8, 2.5×10⁶ PBMCs/cm² were cultured with IL4, IL7 and pepmixes covering all of the above-mentioned RSV antigens for 10-15 days as described in Examples 1 and 2.

### Results

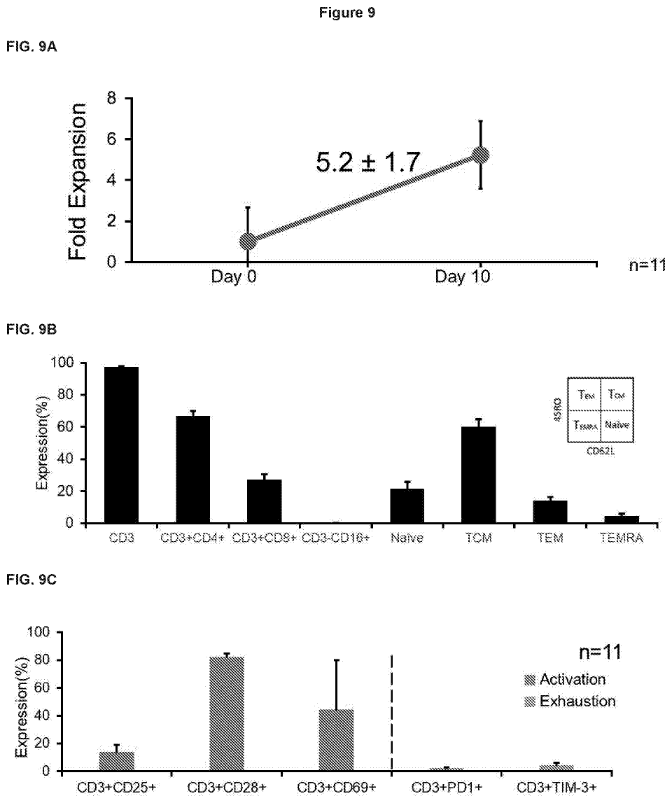

Over 10 days we achieved an average of approximately 5 fold increase in cells (FIG. 9A). We used flow cytometry to immunophenotype the expanded cells as described above. The expanded cells were comprised almost exclusively of CD3+ T cells with a mixture of cytotoxic (CD8+; ˜33%) and helper (CD4+; ˜66%) T cells [FIG. 9B]. Furthermore, the expanded cells displayed a phenotype consistent with effector function and long term memory as evidenced by upregulation of the activation markers CD25, CD69, and CD28, and with minimal PD1 or Tim3 surface expression [FIG. 9C].

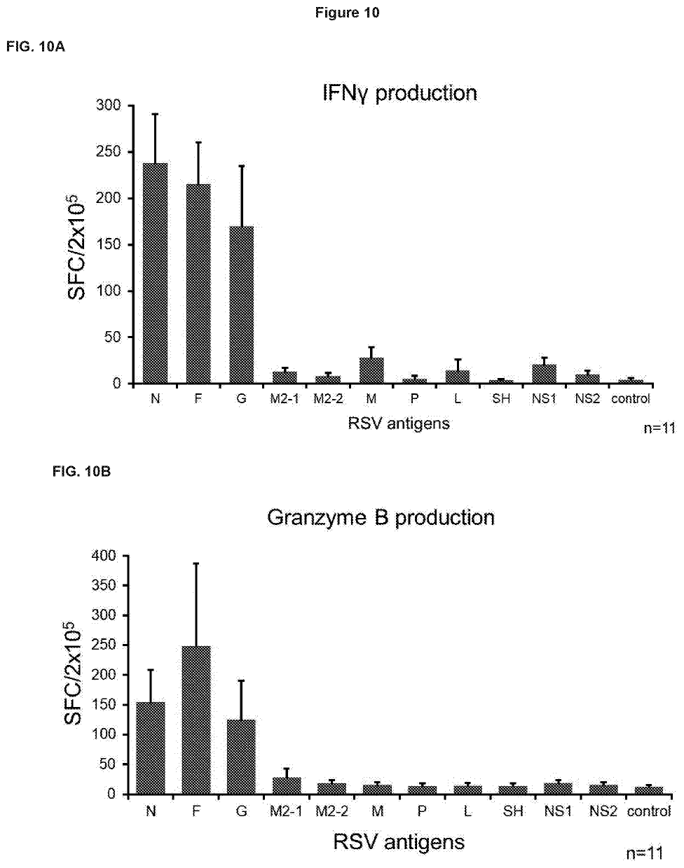

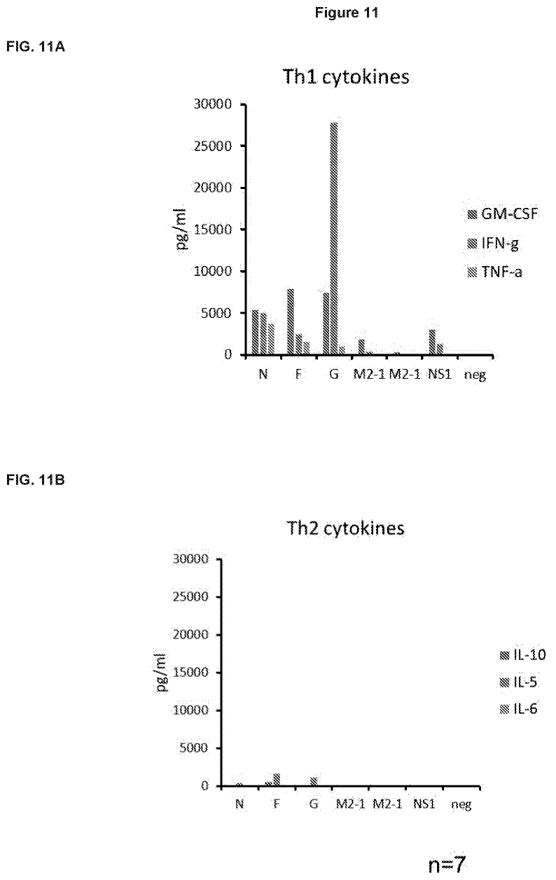

We examined the cytokine profile of our RSV-VSTs following antigen exposure. As shown in FIG. 10A, the VSTs produced large amounts of IFNγ in response to the RSV antigens N, F, and G, as well as weak responses induced by addition of the other RSV antigens. A similar profile of Granzyme B production was seen following pepmix stimulation, suggesting the cytolytic potential of these expanded cells [FIG. 10B]. Furthermore, analysis of Th1 cytokines GM-CSF, IFNγ, and TNFα (FIG. 11A) and the Th2 cytokines IL-5, IL-6, and IL-10 (FIG. 11B) clearly showed that the RSV-specific VSTs were Th1 skewed. Taken together, these data demonstrate the Th1-polarized and polyfunctional characteristics of our multi-R-VSTs.

Thus, the methods disclosed herein result in the rapid expansion of a polyclonal population of activated cytotoxic and helper T cells with no signs of exhaustion suggesting the expansion of VSTs with specificity for the RSV antigens.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

1. Hodinka R L. Respiratory RNA Viruses. Microbiol Spectr. 2016; 4(4).
2. Gill P J, Richardson S E, Ostrow O, et al. Testing for Respiratory Viruses in Children: To Swab or Not to Swab. JAMA Pediatr. 2017; 171(8):798-804.
3. Nair H, Simoes E A, Rudan I, et al. Global and regional burden of hospital admissions for severe acute lower respiratory infections in young children in 2010: a systematic analysis. Lancet. 2013; 381(9875):1380-1390.
4. Shi T, McAllister D A, O'Brien K L, et al. Global, regional, and national disease burden estimates of acute lower respiratory infections due to respiratory syncytial virus in young children in 2015: a systematic review and modelling study. Lancet. 2017; 390(10098):946-958.
5. Paulsen G C, Danziger-Isakov L. Respiratory Viral Infections in Solid Organ and Hematopoietic Stem Cell Transplantation. Clin Chest Med. 2017; 38(4):707-726.
6. Abbas S, Raybould J E, Sastry S, et al. Respiratory viruses in transplant recipients: more than just a cold. Clinical syndromes and infection prevention principles. Int J Infect Dis. 2017; 62:86-93.
7. Hutspardol S, Essa M, Richardson S, et al. Significant Transplantation-Related Mortality from Respiratory Virus Infections within the First One Hundred Days in Children after Hematopoietic Stem Cell Transplantation. Biol Blood Marrow Transplant. 2015; 21(10):1802-1807.
8. Lin R, Liu Q. Diagnosis and treatment of viral diseases in recipients of allogeneic hematopoietic stem cell transplantation. J Hematol Oncol. 2013; 6:94.
9. Renaud C, Xie H, Seo S, et al. Mortality rates of human metapneumovirus and respiratory syncytial virus lower respiratory tract infections in hematopoietic cell transplantation recipients. Biol Blood Marrow Transplant. 2013; 19(8):1220-1226.
10. Shah D P, Shah P K, Azzi J M, et al. Human metapneumovirus infections in hematopoietic cell transplant recipients and hematologic malignancy patients: A systematic review. Cancer Lett. 2016; 379(1):100-106.
11. Shah D P, Shah P K, Azzi J M, et al. Parainfluenza virus infections in hematopoietic cell transplant recipients and hematologic malignancy patients: A systematic review. Cancer Lett. 2016; 370(2):358-364.
12. Chemaly R F, Shah D P, Boeckh M J. Management of respiratory viral infections in hematopoietic cell transplant recipients and patients with hematologic malignancies. Clin Infect Dis. 2014; 59 Suppl 5:S344-351.
13. Beaird O E, Freifeld A, Ison M G, et al. Current practices for treatment of respiratory syncytial virus and other non-influenza respiratory viruses in high-risk patient populations: a survey of institutions in the Midwestern Respiratory Virus Collaborative. Transpl Infect Dis. 2016; 18(2):210-215.
14. Chemaly R F, Aitken S L, Wolfe C R, et al. Aerosolized ribavirin: the most expensive drug for pneumonia. Transpl Infect Dis. 2016; 18(4):634-636.
15. Griffiths C, Drews S J, Marchant D J. Respiratory Syncytial Virus: Infection, Detection, and New Options for Prevention and Treatment. Clin Microbiol Rev. 2017; 30(1):277-319.
16. Walsh E E. Respiratory Syncytial Virus Infection: An Illness for All Ages. Clin Chest Med. 2017; 38(1):29-36.
17. Papadopoulou A, Gerdemann U, Katari U L, et al. Activity of broad-spectrum T cells as treatment for AdV, EBV, CMV, BKV, and HHV6 infections after HSCT. Sci Transl Med. 2014; 6(242):242ra83.
18. Tzannou I, Papadopoulou A, Naik S, et al. Off-the-Shelf Virus-Specific T Cells to Treat B K Virus, Human Herpesvirus 6, Cytomegalovirus, Epstein-Barr Virus, and Adenovirus Infections After Allogeneic Hematopoietic Stem-Cell Transplantation. J Clin Oncol. 2017; 35(31):3547-3557.
19. Aguayo-Hiraldo P I, Arasaratnam R J, Tzannou I, et al. Characterizing the Cellular Immune Response to Parainfluenza Virus 3. J Infect Dis. 2017; 216(2):153-161.
20. Gerdemann U, Keirnan J M, Katari U L, et al. Rapidly generated multivirus-specific cytotoxic T lymphocytes for the prophylaxis and treatment of viral infections. Mol Ther. 2012; 20(8):1622-1632.
21. Tzannou I, Nicholas S K, Lulla P, et al. Immunologic Profiling of Human Metapneumovirus for the Development of Targeted Immunotherapy. J Infect Dis. 2017; 216(6):678-687.
22. Goka E, Vallely P, Mutton K, et al. Influenza A viruses dual and multiple infections with other respiratory viruses and risk of hospitalisation and mortality. Influenza Other Respir Viruses. 2013; 7(6):1079-1087.
23. Goka E A, Vallely P J, Mutton K J, et al. Single, dual and multiple respiratory virus infections and risk of hospitalization and mortality. Epidemiol Infect. 2015; 143(1):37-47.
24. Kouni S, Karakitsos P, Chranioti A, et al. Evaluation of viral co-infections in hospitalized and non-hospitalized children with respiratory infections using microarrays. Clin Microbiol Infect. 2013; 19(8):772-777.
25. Lim F J, de Klerk N, Blyth C C, et al. Systematic review and meta-analysis of respiratory viral coinfections in children. Respirology. 2016; 21(4):648-655.
26. Stefanska I, Romanowska M, Donevski S, et al. Co-infections with influenza and other respiratory viruses. Adv Exp Med Biol. 2013; 756:291-301.
27. Salvatore M, Satlin M J, Jacobs S E, et al. DAS181 for Treatment of Parainfluenza Virus Infections in Hematopoietic Stem Cell Transplant Recipients at a Single Center. Biol Blood Marrow Transplant. 2016; 22(5):965-970.
28. Zenilman J M, Fuchs E J, Hendrix C W, et al. Phase 1 clinical trials of DAS181, an inhaled sialidase, in healthy adults. Antiviral Res. 2015; 123:114-119.
29. Kim Y J, Guthrie K A, Waghmare A, et al. Respiratory syncytial virus in hematopoietic cell transplant recipients: factors determining progression to lower respiratory tract disease. J Infect Dis. 2014; 209(8):1195-1204.
30. Vakil E, Sheshadri A, Faiz S A, et al. Risk factors for mortality after respiratory syncytial virus lower respiratory tract infection in adults with hematologic malignancies. Transpl Infect Dis. 2018; 20(6):e12994.
31. Gerdemann U, Katari U L, Papadopoulou A, et al. Safety and clinical efficacy of rapidly-generated trivirus-directed T cells as treatment for adenovirus, EBV, and CMV infections after allogeneic hematopoietic stem cell transplant. Mol Ther. 2013; 21(11):2113-2121.
32. Heslop H E, Slobod K S, Pule M A, et al. Long-term outcome of EBV-specific T-cell infusions to prevent or treat EBV-related lymphoproliferative disease in transplant recipients. Blood. 2010; 115(5):925-935.
33. Leen A M, Christin A, Myers G D, et al. Cytotoxic T lymphocyte therapy with donor T cells prevents and treats adenovirus and Epstein-Barr virus infections after haploidentical and matched unrelated stem cell transplantation. Blood. 2009; 114(19):4283-4292.
34. Doubrovina E, Oflaz-Sozmen B, Prockop S E, et al. Adoptive immunotherapy with unselected or EBV-specific T cells for biopsy-proven EBV+ lymphomas after allogeneic hematopoietic cell transplantation. Blood. 2012; 119(11):2644-2656.
35. Feucht J, Opherk K, Lang P, et al. Adoptive T-cell therapy with hexon-specific Th1 cells as a treatment of refractory adenovirus infection after HSCT. Blood. 2015; 125(12):1986-1994.
36. Feuchtinger T, Opherk K, Bethge W A, et al. Adoptive transfer of pp65-specific T cells for the treatment of chemorefractory cytomegalovirus disease or reactivation after haploidentical and matched unrelated stem cell transplantation. Blood. 2010; 116(20):4360-4367.
37. Peggs K S, Verfuerth S, Pizzey A, et al. Cytomegalovirus-specific T cell immunotherapy promotes restoration of durable functional antiviral immunity following allogeneic stem cell transplantation. Clin Infect Dis. 2009; 49(12):1851-1860.
38. Chen L, Zanker D, Xiao K, et al. Immunodominant CD4+ T-cell responses to influenza A virus in healthy individuals focus on matrix 1 and nucleoprotein. J Virol. 2014; 88(20):11760-11773.
39. Grant E J, Quinones-Parra S M, Clemens E B, et al. Human influenza viruses and CD8(+) T cell responses. Curr Opin Virol. 2016; 16:132-142.

1. A composition comprising a polyclonal population of cytotoxic T-lymphocytes (CTLs) that recognize a plurality of viral antigens, wherein the plurality of viral antigens comprise at least one first antigen from PIV and at least one second antigen from one or more second viruses.

2. The composition of claim 1, wherein the CTLs are generated by contacting peripheral blood mononuclear cells (PBMCs) with a plurality of pepmix libraries, each pepmix library comprising a plurality of overlapping peptides spanning at least a portion of a viral antigen, wherein at least one of the plurality of pepmix libraries spans a first antigen from PIV-3 and wherein at least one additional pepmix library of the plurality of pepmix libraries spans each second antigen.

3. The composition of claim 1, wherein the CTLs are generated by contacting T cells with dendritic cells (DCs) primed with a plurality of pepmix libraries, each pepmix library comprising a plurality of overlapping peptides spanning at least a portion of a viral antigen, wherein at least one of the plurality of pepmix libraries spans a first antigen from PIV-3 and wherein at least one additional pepmix library of the plurality of pepmix libraries spans each second antigen.

4. The composition of claim 1, wherein the CTLs are generated by contacting T cells with dendritic cells (DCs) nucleofected with at least one DNA plasmid encoding the PIV-3 antigen and at least one DNA plasmid encoding each second antigen.

5. The composition of claim 4, wherein the plasmid encodes at least one PIV-3 antigen and at least one of the second antigens.

6. The composition of any one of claims 1-5, comprising CD4+ T-lymphocytes and CD8+ T-lymphocytes.

7. The composition of any one of claims 1-6, comprising CTLs expressing αβ T cell receptors.

8. The composition of any one of claims 1-7, comprising MHC-restricted CTLs.

9. The composition of any one of claims 1-8, wherein the one or more second viruses is selected from the group consisting of respiratory syncytial virus (RSV), Influenza, human metapneumovirus (hMPV), and a combination thereof.

10. The composition of any one of claims 1-9, wherein the one or more second viruses comprises respiratory syncytial virus (RSV), Influenza, human metapneumovirus, or a combination thereof.

11. The composition of any one of claims 1-9, wherein the one or more second viruses consists of respiratory syncytial virus (RSV), Influenza, human metapneumovirus, or a combination thereof.

12. The composition of any one of claims 1-11, comprising 1, 2, 3, or 4 first antigens.

13. The composition of claim 12, wherein the first antigen is selected from the group consisting of PIV-3 antigen M, PIV-3 antigen HN, PIV-3 antigen N, PIV-3 antigen F, and a combination thereof.

14. The composition of claim 12, comprising the following 4 first antigens: PIV-3 antigen M, PIV-3 antigen HN, PIV-3 antigen N, and PIV-3 antigen F.

15. The composition of any one of the preceding claims, comprising two or three second viruses.

16. The composition of any one of the preceding claims, comprising three second viruses.

17. The composition of claim 16, wherein the three second viruses are influenza, RSV, and hMPV.

18. The composition of any one of claims 1-17, comprising at least two second antigens per each second virus.

19. The composition of any one of claims 1-17, comprising 1, 2, 3, 4, 5, 6, 7, or 8 second antigens.

20. The composition of any one of claims 1-19, wherein the second antigen is selected from the group consisting of influenza antigen NP1, influenza antigen MP1, RSV antigen N, RSV antigen F, hMPV antigen M, hMPV antigen M2-1, hMPV antigen F, hMPV antigen N, and a combination thereof.

21. The composition of claim 19, wherein the second antigen comprises influenza antigen NP1, influenza antigen MP1, or both.

22. The composition of claim 19, wherein the second antigen comprises RSV antigen N, RSV antigen F, or both

23. The composition of claim 19, wherein the second antigen comprises hMPV antigen M, hMPV antigen M2-1, hMPV antigen F, hMPV antigen N, and combinations thereof.

24. The composition of claim 19, wherein the second antigen comprises each of influenza antigen NP1, influenza antigen MP1, RSV antigen N, RSV antigen F, hMPV antigen M, hMPV antigen M2-1, hMPV antigen F, and hMPV antigen N.

25. The composition of any one of claims 1-8, wherein the plurality of antigens comprise PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N.

26. The composition of any one of claims 1-8, wherein the plurality of antigens consist of, or consist essentially of, PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N.

27. The composition of any one of claims 1-26, wherein the CTLs are cultured ex vivo in the presence of both IL-7 and IL-4.

28. The composition of any one of claims 1-27, wherein the multivirus CTLs have expanded sufficiently within 9-18 days of culture such that they are ready for administration to a patient.

29. The composition of any one of claims 1-28, wherein the CTLs exhibit one or more properties selected from:
a. negligible alloreactivity;
b. less activation induced cell death of antigen-specific T cells harvested from a patient than corresponding antigen-specific T cells harvested from the same patient, but not cultured in the presence of both IL-7 and IL-4; and
c. viability of greater than 70%.

30. The composition of any one of claims 1-29, wherein the composition is negative for bacteria and fungi for at least 7 days in culture; exhibit less than 5 EU/ml of endotoxin, and are negative for mycoplasma.

31. The composition of any one of claims 1-30, wherein the pepmixes were chemically synthesized and are, optionally >90% pure.

32. The composition of any one of claims 1-31, wherein the CTLs are Th1 polarized.

33. The composition of any one of claims 1-32, wherein the CTLs are able to lyse viral antigen-expressing targets cells.

34. The composition of any one of claims 1-33, wherein the CTLs do not significantly lyse non-infected autologous or allogenic target cells.

35. A pharmaceutical composition comprising the composition of any one of claims 1-34 formulated for intravenous delivery, wherein the composition is negative for bacteria and fungi for at least 7 days in culture; exhibit less than 5 EU/ml of endotoxin, and are negative for mycoplasma.

36. A method of lysing a target cell comprising contacting the target cell with the composition of any one of claims 1-34 or the pharmaceutical composition of claim 35.

37. The method of claim 36, wherein the contacting occurs in vivo in a subject.

38. The method of claim 36 or 37, wherein the contacting occurs in vivo via administration of the CTLs to a subject.

39. A method of treating or preventing a viral infection comprising administering to a subject in need thereof the composition of any one of claims 1-34 or the pharmaceutical composition of claim 35.

40. The method of claim 38 or 39, wherein between 5×10⁶ and 5×10⁷ CTL/m² administered to the subject.

41. The method of any one of claims 38-40, wherein the subject is immunocompromised.

42. The method of any one of claims 38-41, wherein the subject has acute myeloid leukemia, acute lymphoblastic leukemia, or chronic granulomatous disease.

43. The method of any one of claims 38-42, wherein the subject, prior to receiving the CTLs, received:
a. a matched related donor transplant with reduced intensity conditioning;
b. a matched unrelated donor transplant with myeloablative conditioning;
c. a haplo-identical transplant with reduced intensity conditioning; or
d. a matched related donor transplant with myeloablative conditioning.

44. The method of any one of claims 38-40, wherein the subject
a. has received a solid organ transplantation;
b. has received chemotherapy;
c. has an HIV infection;
d. has a genetic immunodeficiency; and/or
e. has received an allogeneic stem cell transplant.

45. The method of any one of claims 38-44, wherein the composition is administered to the subject a plurality of times.

46. The method of any one of claims 38-45, wherein the administration of the composition effectively treats or prevents a viral infection in the subject, wherein the viral infection is selected from the group consisting of parainfluenza virus type 3, respiratory syncytial virus, Influenza, and human metapneumovirus.

47. The method of any one of claims 38-46, wherein the subject is a human.

48. A composition comprising a polyclonal population of cytotoxic T-lymphocytes (CTLs) that recognize a plurality of viral antigens, wherein the plurality of viral antigens comprise at least one antigen selected from parainfluenza virus type 3 (PIV-3), respiratory syncytial virus, Influenza, and human metapneumovirus.

49. The composition of claim 48, comprising a polyclonal population of cytotoxic T-lymphocytes (CTLs) that recognize a plurality of viral antigens, wherein the plurality of viral antigens comprise at least one antigen from each of parainfluenza virus type 3, respiratory syncytial virus, Influenza, and human metapneumovirus.

50. The composition of claim 48, comprising a polyclonal population of cytotoxic T-lymphocytes (CTLs) that recognize a plurality of viral antigens, wherein the plurality of viral antigens comprise at least two antigen from each of parainfluenza virus type 3, respiratory syncytial virus, Influenza, and human metapneumovirus.

51. The composition of any one of claims 48-50, wherein the plurality of antigens comprise, consist of, or consist essentially of, PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N.

52. A pharmaceutical composition comprising the composition of any one of claims 48-51 formulated for intravenous delivery.

53. The pharmaceutical composition of claim 52, wherein the composition is negative for bacteria and fungi for at least 7 days in culture; exhibit less than 5 EU/ml of endotoxin, and are negative for mycoplasma.

54. A method of lysing a target cell comprising contacting the target cell with the composition of any one of claims 48-51 or the pharmaceutical composition of claim 52.

55. The method of claim 54, wherein the contacting occurs in vivo in a subject.

56. The method of claim 36 or 37, wherein the contacting occurs in vivo via administration of the CTLs to a subject.

57. A method of treating or preventing a viral infection comprising administering to a subject in need thereof the composition of any one of claims 48-51 or the pharmaceutical composition of claim 52.

58. The method of any one of claims 38-44, wherein the composition is administered to the subject a plurality of times.

59. The method of any one of claims 54-58, wherein the administration of the composition effectively treats or prevents a viral infection in the subject, wherein the viral infection is selected from the group consisting of parainfluenza virus type 3, respiratory syncytial virus, Influenza, and human metapneumovirus.

60. The method of any one of claims 54-59, wherein the subject is a human.
